(19) United States
(12) Patent Application Publication
(10) Pub. No.: US 2012/0277409 A1
(43) Pub. Date: Nov. 1, 2012

(54) METAL-POLYSACCHARIDE CONJUGATES: COMPOSITIONS, SYNTHESIS AND METHODS FOR CANCER THERAPY

(75) Inventors: DAVID J. YANG, HOUSTON, TX (US); YEN-SHIAN WU, Kaohsiung City (TW); HSIEN-FAN CHEN, Kaohsiung City (TW); YUN-CHI LO, Kaohsiung City (TW); NING TSAO, Kaohsiung City (TW); CHIH-WEI HSU, Kaohsiung City (TW)

(73) Assignees: BOARD OF REGENTS, THE UNIVERSITY OF TEXAS SYSTEM, Austin, TX (US); TAIWAN HOPAX CHEMS. MFG. CO., LTD., Kaohsiung City (TW)

(21) Appl. No.: 13/443,481

(22) Filed: Apr. 10, 2012

Related U.S. Application Data
(63) Continuation-in-part of application No. 12/009,421, filed on Jan. 18, 2008.
(60) Provisional application No. 60/933,034, filed on Jun. 4, 2007.

(51) Int. Cl. C08B 37/08; C07F 5/00; C07K 14/78; C07F 13/00

(52) U.S. Cl. 530/356; 536/121; 534/14; 534/15

(57) ABSTRACT

The current disclosure, in one embodiment, includes a polysaccharide conjugate. This conjugate has a polysaccharide and at least one liner covalently bound to the polysaccharide. The conjugate also has at least one metal conjugated by said linker. According to another embodiment, the disclosure provides a method of synthesizing a polysaccharide conjugate by covalently bonding a linker to a polysaccharide to obtain an intermediate and by conjugating said intermediate to a metal to form a polysaccharide conjugate. This conjugate has a higher relaxivity, so it is suitable to be used as a contrast medium for hybrid camera.

### CROSS REFERENCES TO THE RELATED APPLICATIONS

This application is a continuation-in-part of prior application Ser. No. 12/009,421, filed Jan. 18, 2008, which claims the benefit of provisional Application No. 60/933,034, filed Jun. 4, 2007.

### FIELD OF INVENTION

The invention generally relates to conjugates of metal and polysaccharides, and specifically relates to polysaccharide conjugates may be used to induce cancer cell death and in cancer therapy, and may be used for the assessment of tumor angiogenesis by MRI.

### BACKGROUND OF THE INVENTION

Angiogenesis processes are involved in the tumor vasculature density and permeability. An increased understanding of these processes as well as cell cycle regulation and cell signaling agents has opened a new era in the treatment of various tumors. Despite the outstanding advances made in the field of angiogenesis, some significant limitations still remain in the treatment of cancer, tumors and other diseases having an angiogenic component via drug agents. One of the most significant limitations at this time relates to the delivery of cytotoxic drugs instead of cytostatic drugs in vivo.

The effectiveness of platinum conjugates against tumor activity has been demonstrated. For instance, cisplatin, a widely used anticancer drug, has been used as alone or in combination with other agents to treat breast and ovarian cancers. Cisplatin, also known as cis-diamminedichloroplatinum (II) (CDDP), is a simple molecule with Pt conjugated to NH₃ molecules. Cisplatin causes cell arrest at S-phase and that leads to mitotic arrest of proliferating cells. Cisplatin also decreases expression of vascular endothelial growth factor (VEGF) during chemotherapy, thus limiting angiogenesis. Cisplatin is effective in the treatment of majority solid tumors. However, clinical applications using cisplatin are limited due to significant nephrotoxicity, myelosuppression, drug resistance, gastrointestinal toxicity, neurotoxicity and other side effects (e.g. vomiting, granulocytopenia and body weight loss). In addition, cisplatin is formulated in bulky vehicles with poor water solubility, which impairs its therapeutic efficacy. Chemical modifications of various platinum conjugates have been made to increase its hydrophilicity, reduce its side effects and improve its therapeutic efficacy; however, these conjugates still present serious drawbacks.

In recent years, MRI (Magnetic Resonance Imaging), CT (Computed Tomography), PET (Positron emission tomography), and SPECT (Single photon emission computed tomography) are all important tools for diagnosing disease status. For instance, cellular imaging by PET and SPECT provide valuable information in monitoring and guiding clinical trials in the treatment of malignancies and other diseases. PET and SPECT agents can show high specific activities because they are created through a nuclear transformation and use carrier free forms of isotopes. On the other hand, CT, MRI, and ultrasonography are prognostic tools because they do not provide cellular target information; thus, assessment of the effectiveness of cancer therapy is not optimal. Currently, hybrid cameras that combine CT or MRI with either PET or SPECT have been designed to enhance sensitivity and quantify drug properties in real time dynamic events. The contrast media for CT or MRI is a blood flow marker; thus, there is a need to develop a novel contrast media with either longer perfusion/diffusion pattern or cellular target information.

### BRIEF SUMMARY OF THE INVENTION

In view of the disadvantages of conventional technology, one object of the present invention is to develop a contrast medium with higher relaxivity that could be used for MRI/PET, MRI/SPECT, CT/PET, or CT/SPECT hybrid imaging and therapy.

To achieve the above objects, the present invention provides a polysaccharide conjugate comprising: a polysaccharide; at least one linker covalently bound to the polysaccharide; and at least one metal conjugated by said linker.

Preferably, the polysaccharide has a molecular weight of between 10,000 daltons and 30,000 daltons.

Preferably, the polysaccharide is selected from the group consisting of: collagen, chondroitin, hyauraniate, chitosan (polyglucosamine), and chitin. More preferably, the polysaccharide comprises chitosan.

Preferably, the polysaccharide has an amino group and the linker is covalently bound to the polysaccharide via said amino group.

Preferably, the linker has a carboxyl group or thiol group, and said metal is conjugated by the carboxyl group or thiol group of the linker.

Preferably, the linker is a monomeric amino acid, chelating agent, or a modifier.

Preferably, the chelating agent is ethylene diamine tetraacetic acid.

Preferably, the linker is the open-ring form of the modifier. More preferably, the modifier is iminothiolane.

Preferably, the metal is selected from the group consisting of: Tc-99m, Cu-60, Cu-61, Cu-67, In-111, Tl-201, Ga-67, Ga-68, As-72, Re-188, Ho-166, Y-90, Lu-177, Sm-153, Sr-89, Gd-157, Gd-158, Bi-212, Bi-213, Fe, Au, Ag, and composite of Au and Ag.

Preferably, the polysaccharide conjugate comprises the polysaccharide ranged from 50% to 80% by weight.

Preferably, the polysaccharide conjugate comprises the linker ranged from 10% to 40% by weight.

Preferably, the polysaccharide conjugate comprises the metal ranged from 10% to 30% by weight.

The present invention also provides a method of synthesizing a polysaccharide conjugate comprising: covalently bonding a linker to a polysaccharide to obtain an intermediate; and conjugating said intermediate to a metal to form a polysaccharide conjugate.

Preferably, the method further comprises drying the polysaccharide conjugate to form a powder.

The polysaccharide conjugate of the present invention exhibits not only prolonged blood circulation but also preferential tumor accumulation. Moreover, the polysaccharide conjugate of the present invention is able to be used as a contrast media for MRI/PET, MRI/SPECT, CT/PET, or CT/SPECT hybrid imaging.

### BRIEF DESCRIPTION OF THE DRAWINGS

The following figures form part of the present specification and are included to further demonstrate certain aspects of the present invention. The invention may be better understood by reference to one or more of these drawings in combination with the description of embodiments presented herein. The patent or application file contains at least one drawing executed in color. Copies of this patent or patent application publication with color drawing(s) will be provided by the Office upon request and payment of the necessary fee.

FIG. 1 illustrates three types of metal-polysaccharide conjugates according to embodiments of the present invention. “AA” designates an amino acid. “M” designates a metal. In FIG. 1A, only one or a few amino acid groups and conjugated metal are present. In FIG. 1B, an intermediate number of amino acid groups and conjugated metal are present. In FIG. 1C the maximum or nearly the maximum possible amino acid groups and conjugated metal are present.

FIG. 2 shows one method (Method A) of synthesis of a platinum analogue (II) and (IV)-polysaccharide conjugate, according to an embodiment of the present invention.

FIG. 3 shows another method (Method B) of synthesis of a polysaccharide-platinum analogue (II) and (IV) conjugate, according to an embodiment of the present invention.

FIG. 4 shows the effect of a platinum-polysaccharide conjugate, according to an embodiment of the present invention, on inhibition of platinum-resistant ovarian cancer cells (2008 c13) at 48 hours (A) and 72 hours(B).

FIG. 5 shows the effect of a platinum-polysaccharide conjugate, according to an embodiment of the present invention, on inhibition of platinum-sensitive ovarian cancer cells (2008) at 48 h (A) and 72 h (B).

FIG. 6 shows the results of flow cytometry showing the apoptotic effects of cisplatin (CDDP) (A) and a platinum-polysacchardide conjugate (PC), according to an embodiment of the present invention, (B) on a platinum-resistant ovarian cancer cell line, 2008-c13, at 48 hours.

FIG. 7 shows the percentages of apoptotic cells detected by flow cytometry in the platinum-resistant ovarian cancer cell line 2008-c13 treated with a platinum-polysaccharide conjugate (PC), according to an embodiment of the present invention, or cisplatin (CDDP) at various concentrations for 48 hours (A) and 72 hours (B).

FIG. 8 shows the percentage of apoptotic cells detected by TUNEL assay in the platinum-resistant ovarian cancer cell line 2008-c13 treated with a platinum-polysaccharide conjugate (PC), according to an embodiment of the present invention, or cisplatin (CDDP) at various concentrations for 48 hours.

FIG. 9 shows the in vivo effects of a platinum-polysaccharide conjugate, according to an embodiment of the present invention, against breast tumor growth at 24 hours (A) and 94 hrs (B) (single dose, Pt 10 mg/kg). The tumors designated DY were taken from an animal administered only chondriotin. The tumors designated DP-A-P were taken from an animal administered the platinum-polysaccharide conjugate. In FIG. 9A, the tumor on the left measured (2.08 cm×2.58 cm×1.96 cm)/2 for a volume of 5.2591 cm³. The tumor on the right measured (2.20 cm×2.37 cm×2.02 cm)/2 for a volume of 5.2661 cm³. In FIG. 9B, the tumor on the left measured (2.99 cm×3.29 cm×2.92 cm)/2 for a volume of 14.3622 cm³. The tumor on the right measured (1.11 cm×1.84 cm×0.86 cm)/2 for a volume of 0.8782 cm³.

FIG. 10 shows H & E staining of tumors to show necrosis at 24 and 94 hrs post-administration of a platinum-polysaccharide conjugate, according to an embodiment of the present invention, or chondroitin alone. FIG. 10A shows a mammary tumor (13762) at 24 hrs administered chondroitin. FIG. 10B shows a mammary tumor (13762) at 24 hrs administered a platinum-polysaccharide conjugate. FIG. 10C shows a mammary tumor (13762) at 94 hrs administered chondroitin. FIG. 10D shows a mammary tumor (13762) at 94 hrs administered a platinum-polysaccharide conjugate.

FIG. 11 shows a Western blot of PARP protein from 2008-c13 cells treated with either platinum-polysaccharide conjugate (PC) or cisplatin (CDDP).

FIG. 12 shows the results of flow cytometric analysis of the cell cycle of 2008-c13 cells platinum-polysaccharide conjugate (PC) or cisplatin (CDDP) after 48 hours.

FIG. 13 shows a Northern blot for p21 transcript (FIG. 13A) and a Western blot for expressed p21 (FIG. 13B) in 2008-c13 cells treated with low doses of platinum-polysaccharide conjugate (PC) or cisplatin (CDDP).

FIG. 14A shows Flow cytometric analysis of the dose-dependent increase of the sub-G₁ fraction after 48 hours-exposure to cisplatin (CDDP) or conjugate (PC or DDAP). At the same doses, PC induced substantially more sub-G₁ cells than did CDDP in platinum-resistant 2008.C13 cells. FIG. 14B shows the percentage of the sub-G₁ fraction in 2008.C13 cells after 48 hours-exposure to CDDP or PC (DDAP).

FIG. 15 shows a TUNEL assay of apoptosis induced by cis-diamminedichloroplatinum(II) (CDDP) and diammine dicarboxylic acid platinum (PC or DDAP) after 48 hours of drug exposure. In FIGS. 15A and 15B, the apoptotic morphology is indicated by brown particles. In FIG. 15C, the percentage of cells with apoptotic morphology. Columns, mean of three independent experiments; bars, SE.

FIG. 16 shows a Western blot analysis of cleaved caspase-3 and specific poly (ADP-ribose) polymerase (PARP) cleavage in 2008.C13 cells treated with cis-diamminedichloroplatinum(II) (CDDP) or diammine dicarboxylic acid platinum (PCor DDAP). GAPDH, glyceraldehyde-3-phosphate dehydrogenase.

FIG. 17A shows the cell-cycle distribution after treatment with cis-diamminedichloroplatinum(II) (CDDP) or diammine dicarboxylic acid platinum (PC or DDAP) for 48 hours in the 2008.C13 cell line. G₁, G₂, M, and S indicate cell phases. FIG. 17B shows a Western blot analysis of p21 and cyclin A expression in 2008.C13 cells after exposure to cis-diamminedichloroplatinum(II) (CDDP) or diammine dicarboxylic acid platinum (PC or DDAP) for 48 hours. GAPDH, glyceraldehyde-3-phosphate dehydrogenase.

FIG. 18 illustrates the structure of the polysaccharide conjugate according to the example 5.

FIG. 19A shows the two-dimensional axial dynamic images by MR scanner of breast tumor-bearing rat after injection of Gd-EDTA-chitosan. FIG. 19B shows a curve of time versus signal intensity for tumor and normal muscle measured using regions of interest (ROIs) defined using T₁-weighted image data. FIG. 19C shows the two-dimensional axial static images by MR scanner of nasopharyngeal tumor-bearing mouse after injection of Gd-EDTA-chitosan at 1 hr. FIG. 19D shows a curve of time versus signal intensity for tumor and normal muscle measured using region of interest from multi-slice multi echo (MSME) T₁-weighted image in MRI scanner post contrast media (CM) Gd-EDTA-chitosan.

FIG. 20A shows the two-dimensional axial dynamic images by MR scanner of breast tumor-bearing rat after injection of Gd-DTPA. FIG. 20B shows a curve of time versus signal intensity for tumor and normal muscle measured using regions of interest (ROIs) defined using T₁-weighted image data. FIG. 20C shows the two-dimensional axial static images by MR scanner of nasopharyngeal tumor-bearing mouse after injection of Gd-DTPA. FIG. 20D shows a curve of time versus signal intensity for tumor and normal muscle measured using region of interest from multi-slice multi echo (MSME) T₁-weighted image in MRI scanner post contrast media (CM) Gd-EDTA.

FIG. 21 shows the results of MRI (1.5 T) of a normal pig (20 kg) administered with Gd-EDTA-chitosan (0.13 mmol/kg, iv) at 15 min post-injection.

FIG. 22 shows the results of MRI (1.5 T) (angiography) of a normal pig (20 kg) administered with Gd-EDTA-chitosan (0.13 mmol/kg, iv) at 15 min post-injection.

FIG. 23 shows the results of MRI (1.5 T) of a normal pig (20 kg) administered with Gd-EDTA-chitosan (0.13 mmol/kg, iv) at 15 min post-injection.

FIG. 24 shows the result of MRI (1.5 T) of a melanoma-bearing pig (23 kg) administered with Gd-EDTA-chitosan (0.13 mmol/kg, iv) at 5 min post-injection.

FIG. 25 shows the result of MRI (1.5 T) of a melanoma-bearing pig (23 kg) administered with Gd-EDTA-chitosan (0.13 mmol/kg, iv) at 30 min post-injection.

FIG. 26 shows the result of MRI (1.5 T) of a melanoma-bearing pig (23 kg) administered with Gd-EDTA-chitosan (0.13 mmol/kg, iv) at 60 min post-injection.

FIG. 27 shows time-activity curve of tumor uptake (signal intensity) versus time of Gd-EDTA-chitosan in a melanoma-bearing pig.

FIG. 28 shows the percentage (%) of tumor enhancement.

FIG. 29A shows planar scintigraphy of breast tumor-bearing rats after treatment with ^{99m}Tc-EDTA (3 mg, 300 μCi) for 0.5 hr. FIG. 29B shows planar scintigraphy of breast tumor-bearing rats after treatment with ^{99m}Tc-EDTA-chitosan (1 mg, 300 μCi) for 0.5 hr. FIG. 29C shows planar scintigraphy of breast tumor-bearing rats after treatment with ^{99m}Tc-EDTA-chitosan (1 mg, 300 μCi) for 1 hr. Besides, T/M respresents tumor-to-muscle count densty (cpm/pixel) ratio.

FIG. 30 shows the results of phantom assay of Gd-EDTA-chitosan at various concentrations.

FIG. 31 shows a curve of time versus temperature achieved by Gd-EDTA-chitosan or DG-DTPA using ultrasound bath at 40 KHz.

FIG. 32 shows the images of Fourier transform infrared spectrum of chitosan and thiol compound-chitosan conjugate (chitosan-SH).

### DETAILED DESCRIPTION

The present invention provides a polysaccharides conjugate that can be used as a contrast medium (or macrocontrast medium) for hybrid camera such as nuclear scan or MRI. Moreover, the polysaccharide conjugate used as a contrast medium exhibits longer circulation time so that it is able to provide better small lesion detection and hemodynamic changes.

The polysaccharides conjugate of the present invention comprise a polysaccharide; at least one linker covalently bound to the polysaccharide; and at least one metal conjugated by said linker.

The present invention, in certain embodiments, includes metal-polysaccharides conjugates, methods for their synthesis, and uses thereof, including inducing cancer cell death, treatment of cancer, and diagnosing disease status. In particular, the conjugate may include a polysaccharide with at least one linker attached. This linker may then conjugate the metal. In a preferable embodiment, the linker is a monomeric amino acid, a chelating agent, or a modifier. In selected embodiments that the linker is a monomeric amino acid, the linker may conjugate the metal via an O-group rather than a N-group. In some embodiments, the metal may be a transition metal. In many embodiments, there may be multiple monomeric amino acids attached, which allows for conjugation of multiple metal groups. The conjugates may be of any size, but in certain embodiments, the conjugate may be designed so that each molecule is at least 10,000 daltons, for example between 10,000 and 50,000 daltons, to limit excretion through the kidneys. In a particular embodiment, the polysaccharide conjugate may have a molecular weight of between about 20,000 daltons to about 50,000 daltons, more particularly it may be between about 26,000 to about 30,000 daltons.

The polysaccharide selected may be any polysaccharide, but polysaccharides involved in vascular uptake may be particularly useful. In particular, adhesive molecules, such as collagen, chondroitin, hyauraniate, chitosan, and chitin may be well suited for use as the polysaccharide. In one particular embodiment, it may be chondroitin A. Although the present invention is not limited to a particular mode of action, such polysaccharides may facilitate uptake through the vasculature and delivery to cancer cells. This may be particularly true in areas undergoing angiogenesis, such as most tumors. The end product molecular weight range of 20,000-50,000 daltons will help achieve vascular-based therapy.

The amino acid may be attached to the polysaccharide in any stable manner, but in many embodiments it will be covalently bonded to the polysaccharide. The amino acid may be in monomeric form, such that individual monomers are attached separately to the polysaccharide. The amino acid may have an O-group available for conjugation of the metal, in particular, it may have two O-groups available. The metal may be conjugated by a single monomeric amino acid, or via two or more monomeric amino acids. Example amino acid monomers that may be used alone or in combination include: glutamic acid, aspartic acid, glutamic acid combined with alanine, glutamic acid combined with asparagine, glutamic acid combined with glutamine, glutamic acid combined with glycine, and aspartic acid combined with glycine. Due to bond distance between two carboxylic acid and better tumor uptake specificity, aspartic acid is preferred. The amino acids may be in L-form, or D-form, or a racemic mixture of L- and D-forms. Amino acid in L-form is preferred for optimal tumor uptake. Aspartic acid may be selected because a single aspartic acid monomer is able to conjugate a metal on its own. Additionally, aspartic acid is not produced by mammalian cells, but is a necessary nutrient, making it likely to be taken up by rapidly growing tumor cells.

The amino acid may comprise between about 10% to about 50%, by weight of the polysaccharide conjugate.

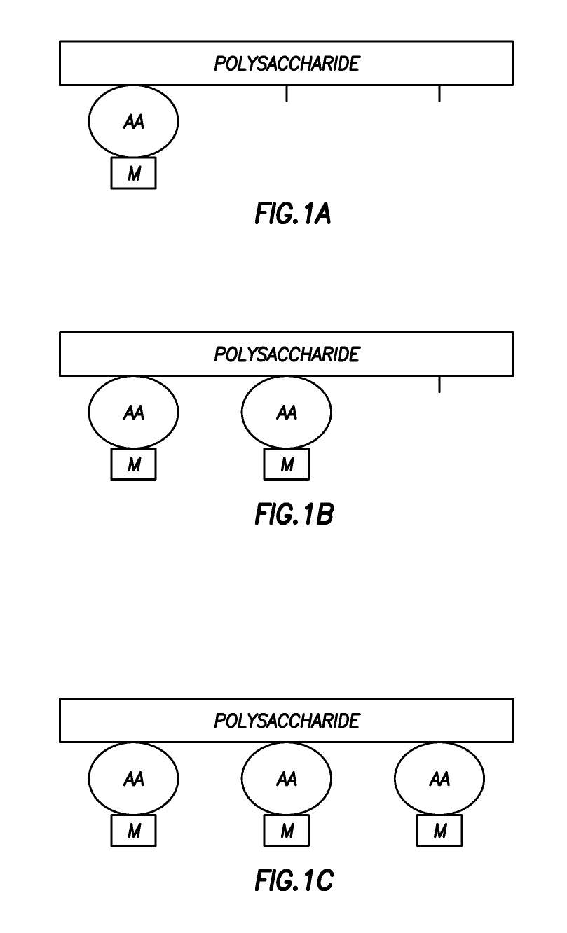

The degree of saturation of amino acid attachment points on the polysaccharide may vary. For example, as shown in FIG. 1A, only one amino acid may be attached. Very low degrees of saturation, such as 5% or less, 10% or less, or 20% or less may also be achieved. FIG. 1B illustrates a conjugate with an intermediate degree of saturation, such as approximately 30%, approximately 40%, approximately 50%, or approximately 70%. FIG. 1C illustrates a conjugate with very high degrees of saturation, such as 80% or greater, 90% or greater, 95% or greater, or substantially complete saturation. Although in FIG. 1 each amino acid has a conjugated metal, in many actual examples, there will be degrees of saturation of the available amino acids by the metal, such as less than 5%, 10% or 20%, approximately 30%, approximately 40%, approximately 50%, or approximately 70%, greater than 80%, 90%, 95%, or substantially complete saturation.

The metal may be any metal atom or ion or compound containing a metal that can be conjugated by the O-groups of the amino acid monomers. In specific embodiments, the metal may be a transition metal, such as platinum, iron, gadolinium, rhenium, manganese, cobalt, indium, gallium, or rhodium. The metal may be a therapeutic metal. It may be part of a larger molecule, such as a drug. The metal may be conjugated to the polysaccharide-amino acid backbone via O-groups of the amino acid monomers.

In one embodiment, the metal may be between 15 percent to about 30 percent, by weight of the polysaccharide conjugate.

In one example embodiment, the conjugate includes chondroitin A covalently bonded to aspartic acid monomers, which conjugate platinum in a platinum-containing compound. In one variation the platinum may be platinum (II) and in another variation the platinum may be platinum (IV).

The conjugate may be water soluble. For example, it may have a solubility of at least approximately 20 mg (metal equivalent)/mL water. The conjugate may be provided in a variety of forms, such as an aqueous solution or a powder. The conjugate and its formulations may be sterilized. For example, it may be provided as a sterilized powder.

The conjugate may be synthesized, according to one embodiment of the invention, by separately covalently bonding one or more amino acid monomers to a polysaccharide. Then a metal may be provided for conjugation by the amino acid monomers. According to another embodiment of the invention, the metal may be conjugated to the amino acid monomers, then one or more of the amino acid monomers may be covalently bonded to the polysaccharide.

The conjugates of the present invention may be used to kill cancer or tumor cells and thus may treat cancer or tumors. The conjugates may target tumors, particularly solid tumors. This may be verified, for example, through radiolabeled variations of the compounds, such as a polysaccharide-amino acid backbone conjugated to ^{99m}Tc, which allows gamma imaging. Cytotoxic agents with a metal component may be conjugated to the polysaccharide-amino acid backbone to reduce their cytotoxic effects. For example, the cytotoxic agents maybe released gradually from the polyssaccharide, decreasing acute systemic toxicity. Additionally, the therapeutic index (toxicity/efficacy) of drugs with poor water solubility or tumor targeting capacity may be increased by conjugating those drugs to the polysaccharide-polymer backbone.

In specific example embodiments, platinum-containing conjugates may be able to inhibit cancer cell growth at lower doses than cisplatin. Further, platinum-containing conjugates may also be able to inhibit cell growth of cisplatin-resistant cancer cells, particularly ovarian cancer cells.

Any type of cancer or tumor cell may be killed or have its growth inhibited by selected conjugates of the present invention. However, solid tumors may respond best to these conjugates. Example cancers that may be susceptible to certain conjugates of the present invention include: ovarian cancer, cisplatin-resistant ovarian cancer, pancreatic cancer, breast cancer, sarcoma, uterine cancer, and lymphoma.

In addition to cancer, certain conjugates of the present invention may be able to target and inhibit cells involved in the development and progression of the following diseases: HIV, autoimmune diseases (e.g. encephalomyelitis, vitiligo, scleroderma, thyroiditis, and perforating collagenosis), genetic diseases (e.g xeroderma pigmentosum and glucose-6-phosphate dehydrogenase deficiency), metabolic diseases (e.g. diabetes mellitus), cardiovascular diseases, neuro/psychiatric diseases and other medical conditions (e.g. hypoglycemia and hepatic cirrhosis).

As stated above, the polysaccharide selected may be any polysaccharide. However, in the consideration of ease of attaching linker, the polysaccharide with an amino group may be particularly useful. In particular, the linker is covalently bound to the amino group of the polysaccharide. Besides, the polysaccharide preferably has a molecular weight of between 10,000 daltons and 30,000 daltons. In particular, the polysaccharide has a molecular weight of between 10,000 daltons and 15,000 daltons. Besides, the polysaccharide may be present in an amount of between 50% to 80% by weight of the polysaccharide conjugate.

In selected embodiments that the linker is a chelating agent or a modifier, the polysaccharide conjugate may have a molecular weight of between 10,000 and 40,000.

In a preferable embodiment, the linker conjugates the metal via its carboxyl group or thiol group.

In some embodiments, the linker may be a chelating agent or a modifier. There is no particular limitation on the chelating agent, as long as the chelating agent is able to chelate a metal such as three to five valent metal. In the present invention, the chelating agent may chelate the metal via N-, S-, O-, P-group, or combination thereof In general, the chelating agent used in the present invention is ethylene diamine tetraacetic acid (EDTA).

The “modifier” used herein refers to a compound for modifying the functional group of the polysaccharide. There is no particular limitation on the modifier, as long as the polysaccharide would have a thiol group after being reacted with the modifier. The modifier includes, but not limited to, iminothiolane (such as 2-iminothiolane). In a specific example, the polysaccharide (such as chitosan) is modified with 2-iminothiolane to obtain a conjugate of polysaccharide and open-ring form of 2-iminothiolane (i.e. polysaccharide-thiol compound). The reaction scheme is shown as below. Subsequently, the metal is conjugated by the thiol group of the polysaccharide-thiol compound to form a polysaccharide conjugate.

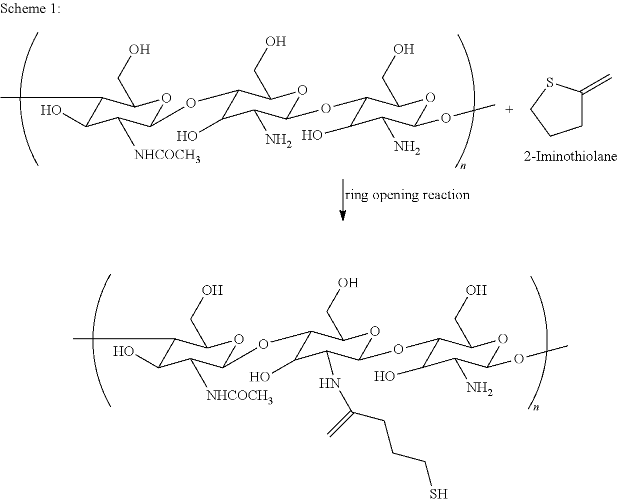

The linker may be present in an amount of between 10% to 40% by weight of the polysaccharide conjugate.

In some embodiments, the metal is selected from the group consisting of: Tc-99m, Cu-60, Cu-61, Cu-67, In-111, Tl-201, Ga-67, Ga-68, As-72, Re-188, Ho-166, Y-90, Lu-177, Sm-153, Sr-89, Gd-157, Gd-158, Bi-212, Bi-213, Fe, Au, Ag, and composite of Au and Ag. Of these metals, Tc-99m, In-111, Tl-201, Ga-67, Ga-68, Re-188, Y-90, Lu-177, Gd-157, or Gd-158 is preferable. In addition, the composite of Au and Ag used in the present invention is preferably one disclosed in Taiwanese patent application No. 100111493, which is incorporated herein by reference.

The metal may be present in an amount of between 10% to 30% by weight of the polysaccharide conjugate. In particular, the metal may be present in an amount of between 12% to 26% by weight of the polysaccharide conjugate.

The present invention also provides a method of synthesizing a polysaccharide conjugate comprising: covalently bonding a linker to a polysaccharide to obtain an intermediate; and conjugating said intermediate to a metal to form a polysaccharide conjugate.

In a preferable embodiment, the method further comprises drying the polysaccharide conjugate to form a powder.

### EXAMPLES

The following examples are included to demonstrate specific embodiments of the invention. It should be appreciated by those of skill in the art that the techniques disclosed in the examples that follow represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention.

### Example 1

### Synthesis of Platinum Analogue (II) and (IV)-Polysaccharide Conjugate

### Method A.

Cis-1,2-Diaminocyclohexane sulfatoplatinum (II) (cis-1,2-DACH-Pt.SO₄) was synthesized via a two-step procedure. In the first step, cis-1,2-DACH-PtI₂ complex was synthesized by mixing a filtered solution of K₂PtCl₄(5.00 g, 12 mmol) in 120 ml of deionized water with KI (20.00 g in 12 ml of water, 120 mmol) and was allowed to stir for 5 min. To this solution one equivalent of the cis-1,2-DACH(1.37 g, 1.487 ml, 12 mmol) was added. The reaction mixture was stirred for 30 min at room temperature. The obtained yellow solid was separated by filtration and then washed with a small amount of deionized water. The final product was dried under vacuum, which yielded cis-1,2-DACH-PtI₂ (6.48 g, 96%). In the second step, cis-1,2-DACH-PtI₂ (without further purification from step 1, 6.48 g, 11.5 mmol) was added as a solid to an aqueous solution of Ag₂SO₄ (3.45 g, 11 mmol). The reaction mixture was left stirring overnight at room temperature. The AgI was removed by filtration and the filtrate was freeze dried under vacuum, which yielded yellow cis-1,2-DACH-Pt(II)SO₄ (4.83 g, 99%). Elemental analysis showed Pt: 44.6% (w/w).

To a stirred solution of chondroitin (1 g, MW. 30,000-35,000) in water (5 ml), sulfo-NHS (241.6 mg, 1.12 mmol) and 3-ethylcarbodiimide1-ethyl-3-(3-dimethylaminopropyl)carbodiimide-HCl (EDC) (218.8 mg, 1.15 mmol) (Pierce Chemical Company, Rockford, Ill.) were added. L-aspartic acid sodium salt (356.8 mg, 1.65 mmol) was then added. The mixture was stirred at room temperature for 24 hours. The mixture was dialyzed for 48 hours using a Spectra/POR molecular porous membrane with cut-off at 10,000 (Spectrum Medical Industries Inc., Houston, Tex.). After dialysis, the product was filtered and freeze dried using lyophilizer (Labconco, Kansas City, Mo.). The product, aspartate-chondroitin (polysaccharide), in the salt form, weighed 1.29 g. A similar technique was used to prepare condroitin having glutamic acid and alanine, glutamic acid and asparagine, glutamic acid and glutamine, glutamic acid and glycine, and glutamic acid and one aspartic acid conjugated with alanine, asparagine, glutamine, and glycine.

Cis-1,2-DACH-Pt (II) SO₄ (500 mg, 1.18 mmol) was dissolved in 10 ml of deionized water, and a solution of aspartate-chondroitin (1.00 g in 15 ml of deionized water) was added. The solution was left stirring for 24 hr at room temperature. After dialysis (MW: 10,000) and lyophilization, the yield of cis-1,2-DACH-Pt (II)-polysaccharide was 1.1462 g.

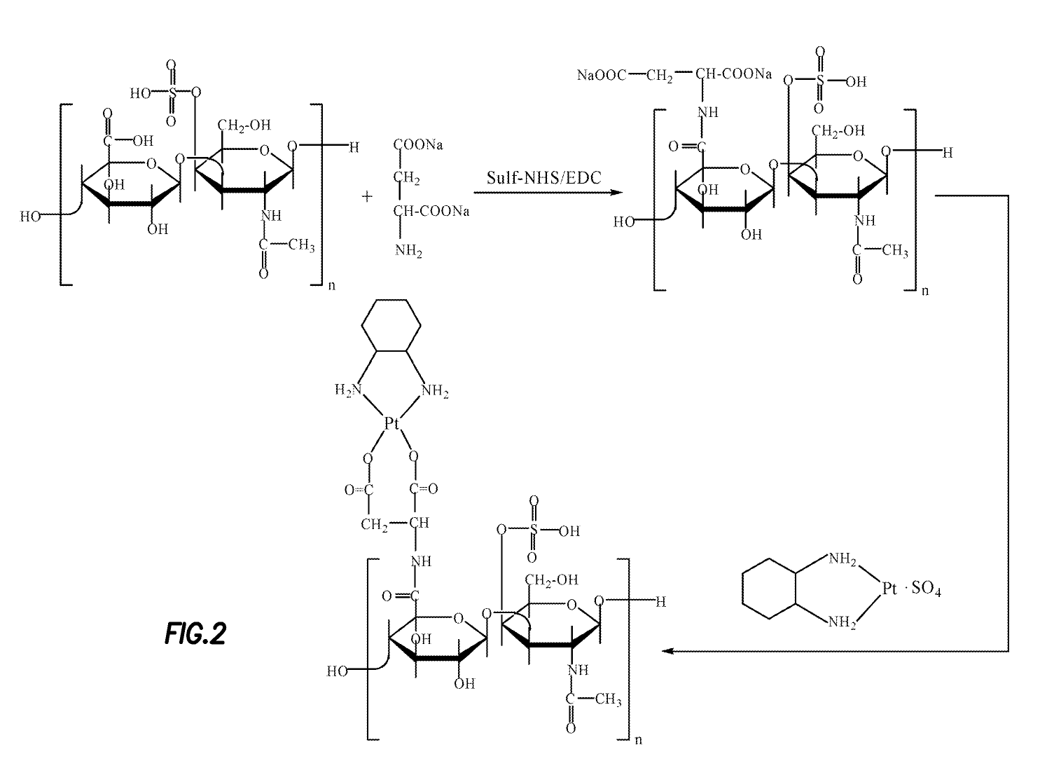

The platinum-polysaccharide conjugate, Cis-1,2-DACH-dichloro-Pt (IV)-aspartate-chondroitin (PC) was synthesized as follows: the above solution was added dropwise 2.5 ml of 30% aqueous hydrogen peroxide. After 24 hr, HCl (75 ml of 0.02 N) was added and left stirring for 24 hr at room temperature, dialyzed (MW: 10,000) by deionized water for overnight and freeze dried under vacuum. The final product obtained was 1.15 g. Elemental analysis showed Pt: 21.87% (w/w). The synthetic scheme is shown in FIG. 2.

### Method B.

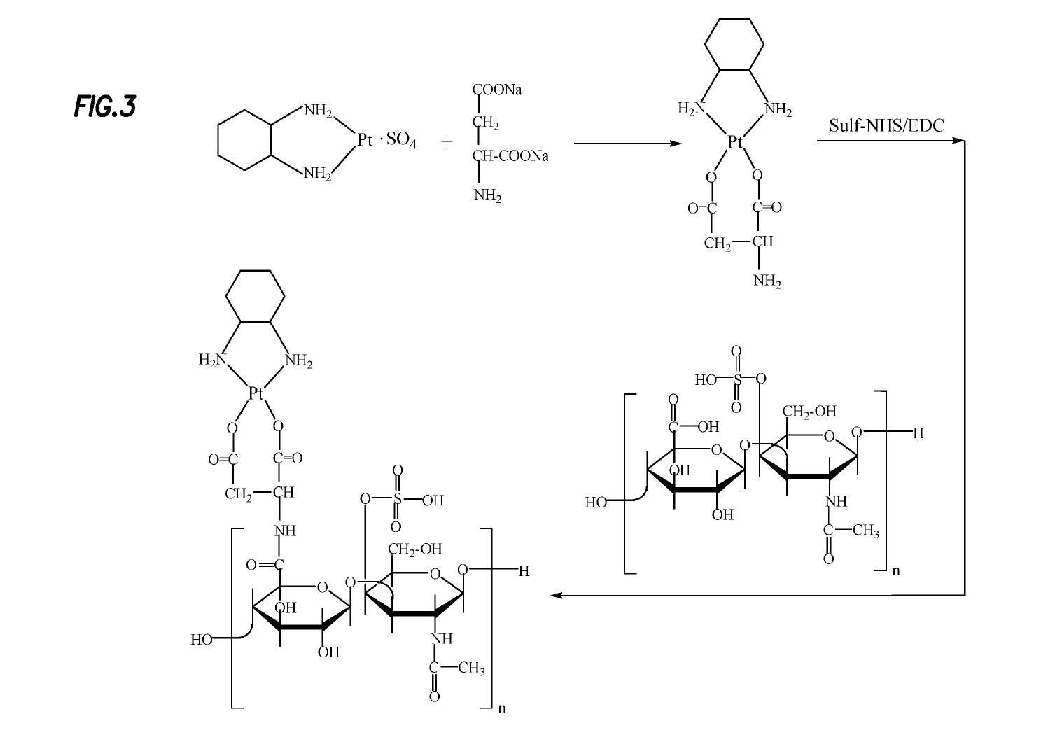

The Cis-1,2-DACH-Pt (II) SO₄ or Cis-1,2-DACH-dichloro-Pt (IV) (500 mg, 1.18 mmol) was dissolved in 10 ml of deionized water, and a solution of aspartic acid (67 mg, 0.5 mmol) in 2 ml of deionized water was added. The solution was left stirring for 24 hr at room temperature. After dialysis and lyophilization, the cis-1,2-DACH-Pt-aspartate was reacted with chondroitin (1 g, MW. 30,000-35,000) in water (5 ml), sulfo-NHS (241.6 mg, 1.12 mmol) and 3 -ethylcarbodiimidel-ethyl-3-(3-dimethylaminopropyl)carbodiimide-HCl (EDC) (218.8 mg, 1.15 mmol) (Pierce Chemical Company, Rockford, Ill.). The synthetic scheme is shown in FIG. 3.

### Example 2

### In Vitro Cell Culture Assay

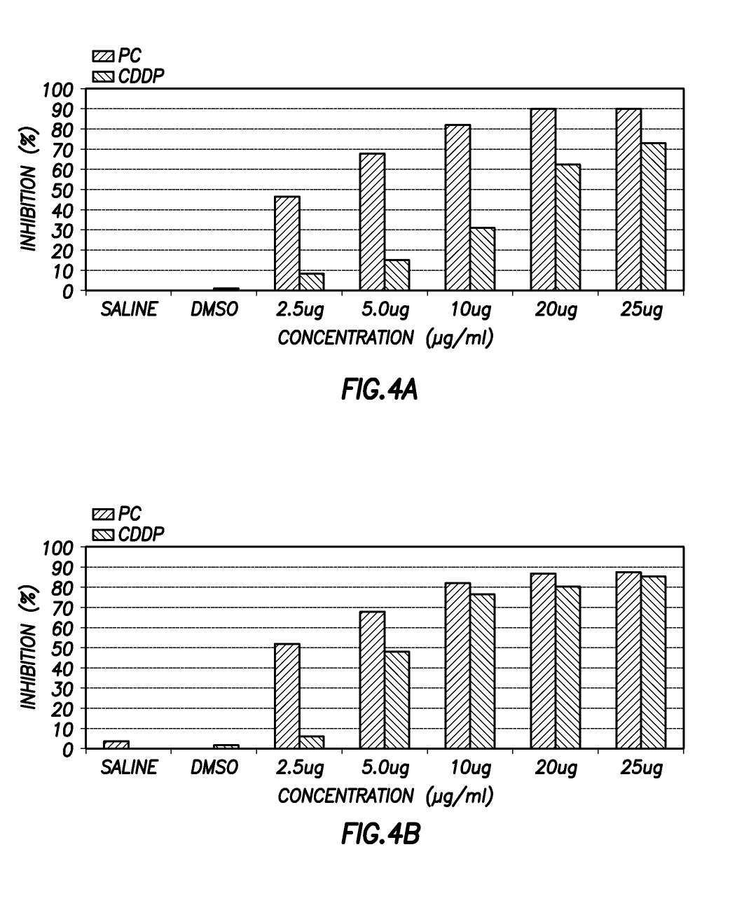

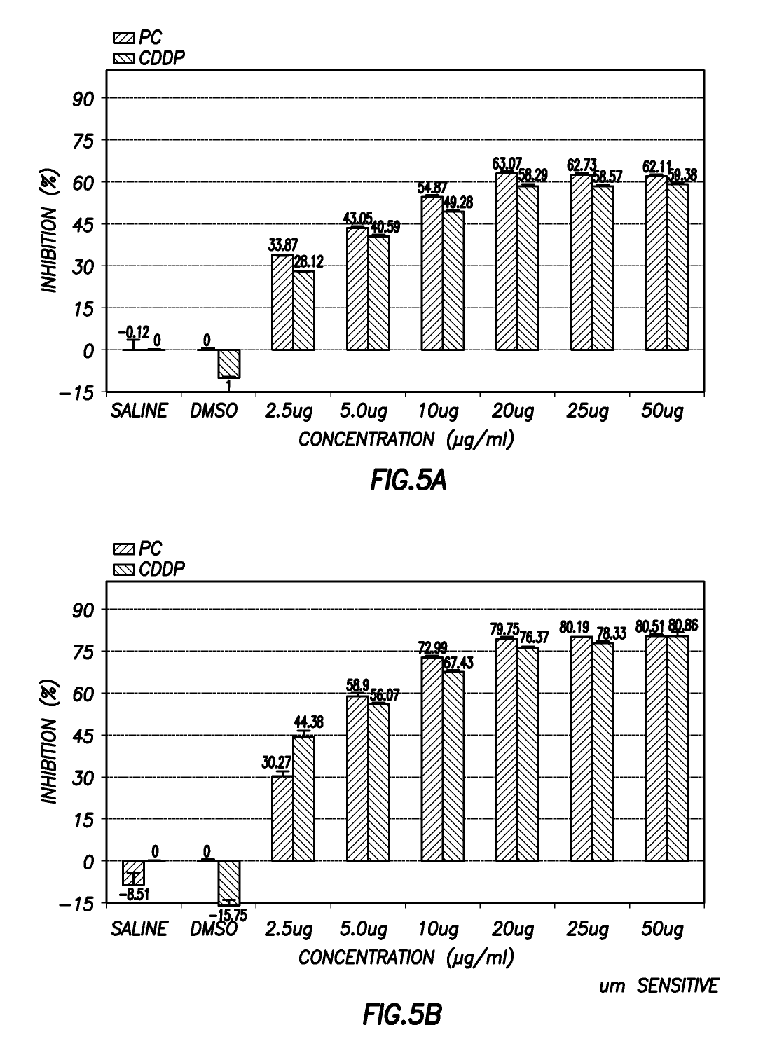

To evaluate cytotoxicity of cisplatin and platinum (II)-polysaccharide conjugate (PC) prepared as described above using Method A against mammary tumor cells, two human tumor cell lines were selected: the 2008 line and its platinum-resistant subline, 2008-c13. All cells were cultured at 37° C. in a humidified atmosphere containing 5% CO₂ in RPMI 1640 medium supplemented with 10% fetal bovine serum and glutamine (2 mM). 2008 or 2008.C13 cells were seeded into 96-well plates (4,000 cells/well) and maintained in RPMI 1640 medium for 24 hours. Next, cells were treated with PC or CDDP at concentrations of 2.5, 5, 10, 20, 25, and 50 μg/mL for 48 and 72 hours. Controls were treated with DMSO or PBS. After cells were treated, their growth and viability were determined by incubating the cells for 1 to 2 hours at 37° C. with 20 μL of tetrazolium substrate. Absorbence was measured at 450 nm using a 96-well Synergy HT-microplate reader (Biotek, Winooski, Vt.) The rate of cell growth inhibition was expressed as a percentage as follows: %=100−(OD_{controls}−OD_{treated cells})/OD_{controls}. The experiments were repeated separately three times. Methylene tetrazolium (MTT) dye assay determined the amount of viable cells. Cellular protein content was determined by Lowry assay. The drug concentration that inhibits 50% of cell growth (IC-50) was then determined Data are expressed as the percentage differences compared with controls (OD of cells after treatment/OD of cells without treatment). An illustrated cell inhibition curves are shown in FIGS. 4 and 5.

The findings showed that the sensitivity of cells to exposure to the IC-50 of platinum-polysaccharide conjugate (PC) was 5.7 times greater than that to the exposure to the IC-50 of cisplatin (CDDP) (FIG. 4) in the platinum-resistant ovarian cancer cell line but not in the platinum-sensitive ovarian cancer line (2008) (FIG. 5). In particular, concentrations of platinum-polysaccharide conjugate (PC) at 2.5 and 5 μg/mL enhanced tumor killing by 5.9 and 4.6 times, respectively, at 48 hours compared with cisplatin (CDDP) (FIG. 4A) and by 9.3 and 1.5 times, respectively, at 72 hours compared with cisplatin (CDDP) (FIG. 4B). The data indicated that low doses of platinum-polysaccharide conjugate (PC) significantly inhibit cell growth of platinum-resistant ovarian cancer cells.

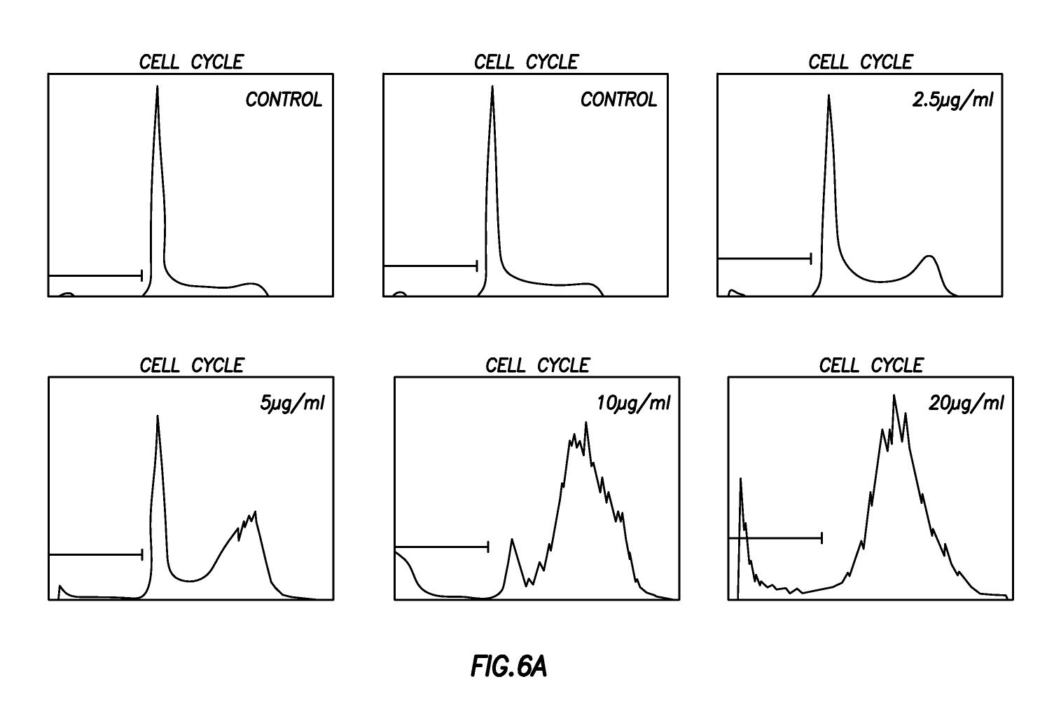

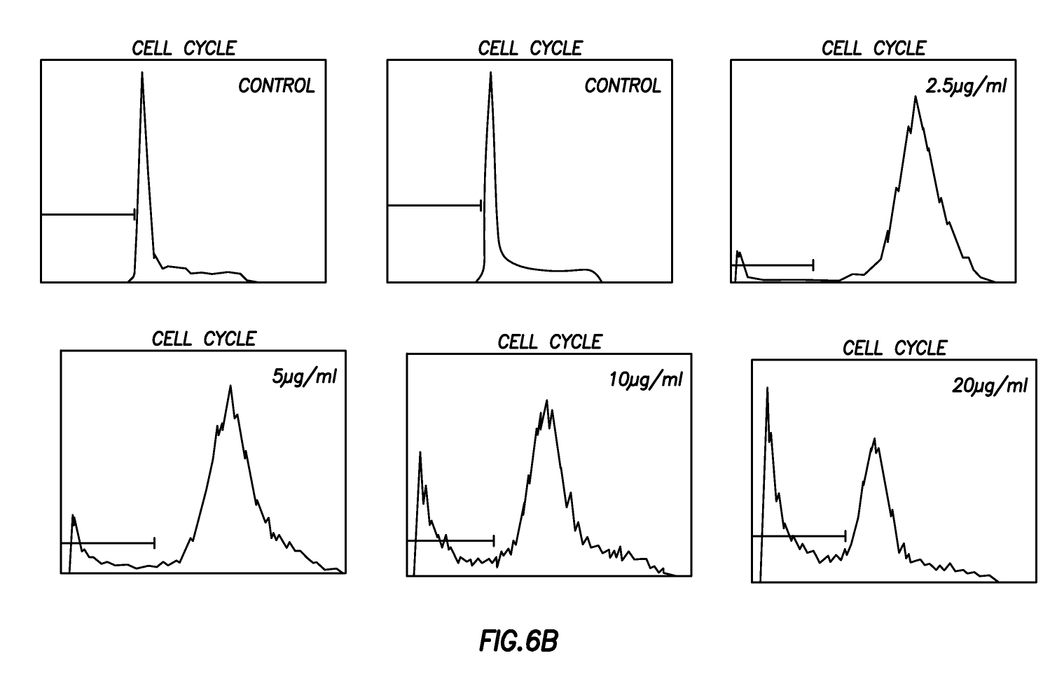

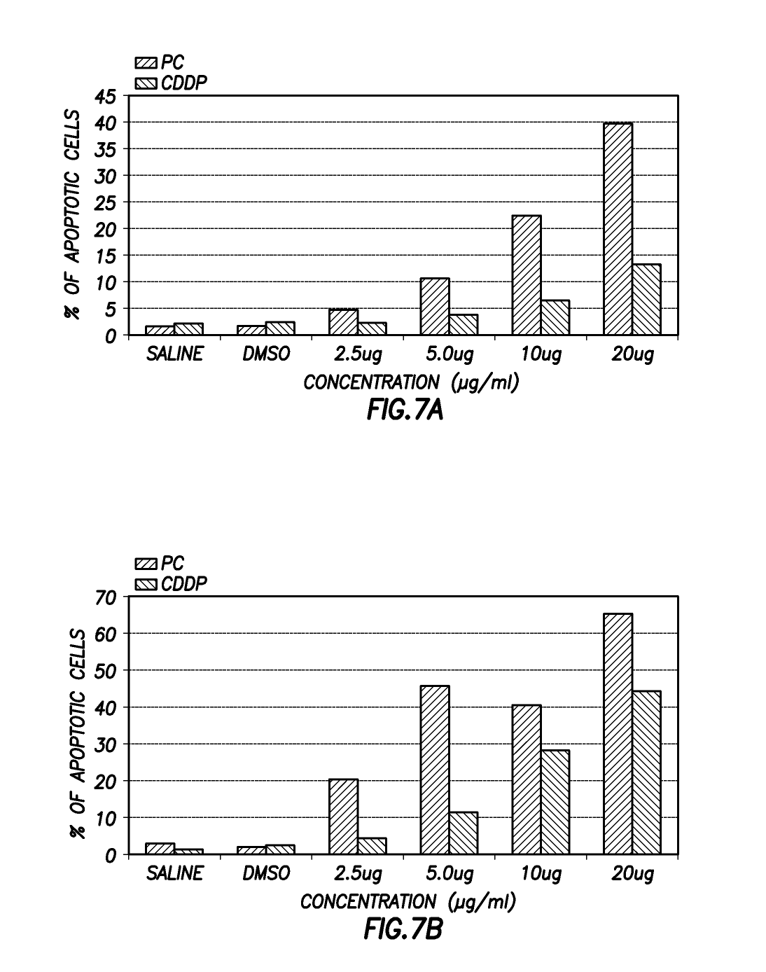

To determine the effectiveness of platinum-polysaccharide conjugate (PC) against platinum-resistant ovarian cancer cells, 2008-c13 cells (0.5×10⁻⁶) were treated with platinum-polysaccharide conjugate (PC). The cells were trypsinized and centrifuged at 2500 rpm for 5 min. After being washed with 1×PBS two times, cells were fixed with 70% ethanol overnight, washed twice with 1×PBS, and resuspended in 1 mL of propidium iodide (PI) solution (1×10⁶ cells/mL). RNase (20 μg/mL) solution was added followed by 1 mL of propydium iodide solution (PI, 50 μg/mL in PBS). Samples were incubated at 37° C. for 15 min, and PI fluorescence was analyzed using a EPIS XL analyzer. Compared to cisplatin (CDDP), platinum (IV)-polysaccharide conjugate at low concentrations (2.5 and 5 μg/mL) significantly enhanced the apoptotic effect on platinum-resistant ovarian cancer cells (FIGS. 6-7).

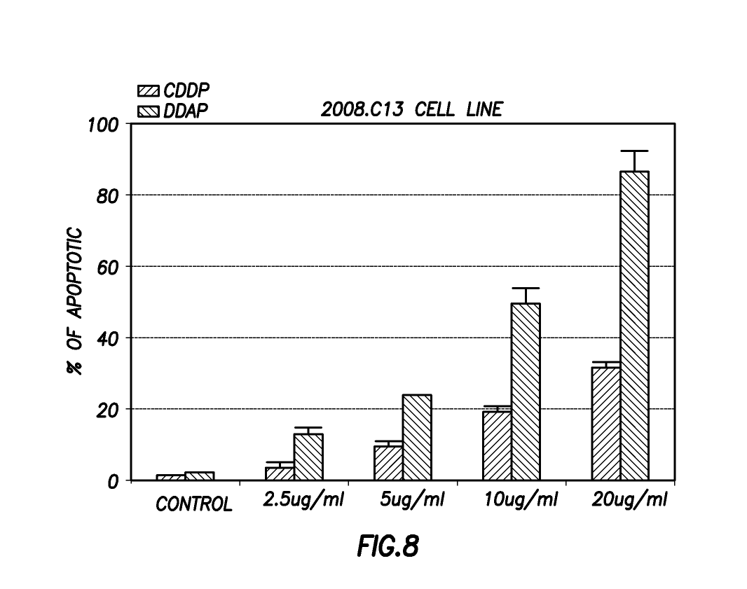

These results were confirmed by TUNEL assay, which, after 48 hours of treatment, shows a clear dose-dependent increase of apoptotic cells was detected after exposure to both drugs. However, when compared at each dose, platinum-polysaccharide conjugate (PC) treated group had many more cells experiencing apoptosis (P<0.05) (FIG. 8).

### Example 3

### Evaluation of Anticancer Effect Using Breast Tumor-Bearing Rat Model

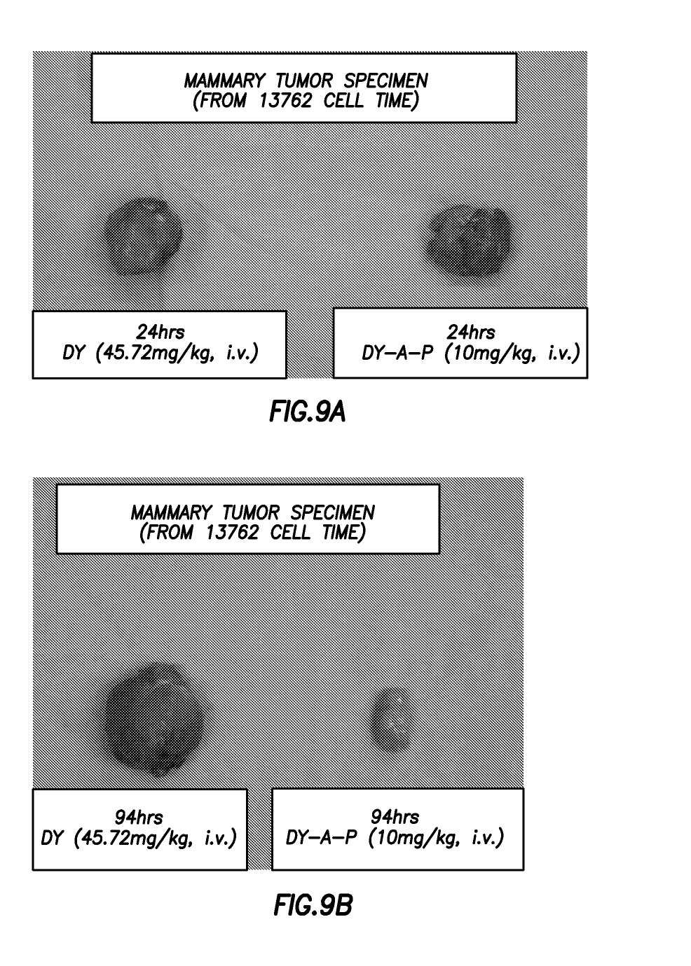

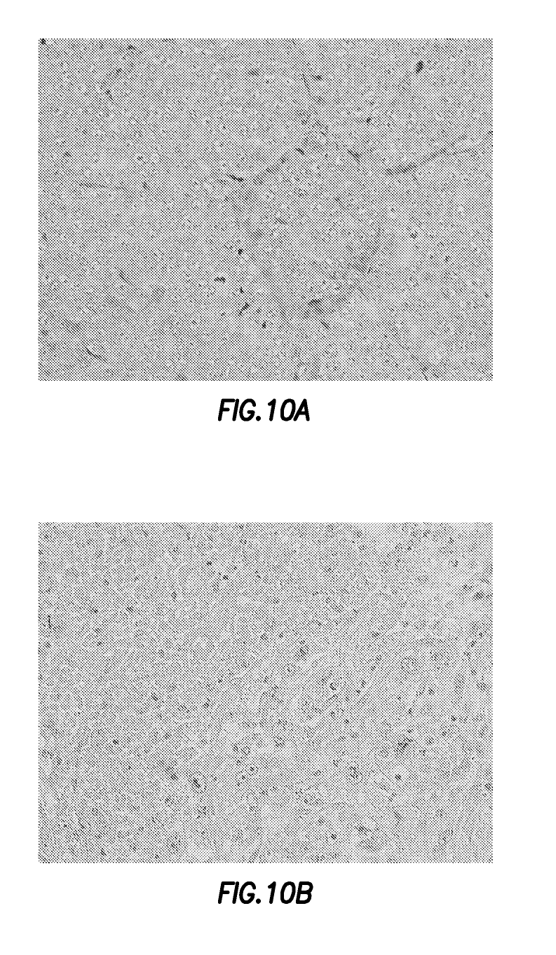

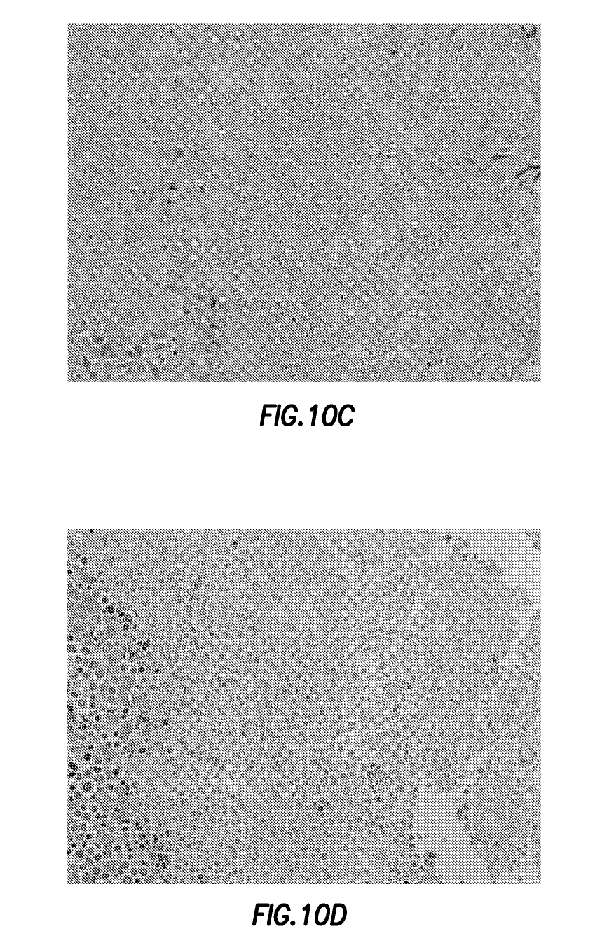

Female Fischer 344 rats (125-175 g) were inoculated with breast cancer cells (13762NF, 10⁶ cells/rat, s.c. in the hind leg). After 15-20 days and a tumor volume of 1 cm, the breast tumor-bearing rats were administered either the platinum-chondroitin (Platinum-polysaccharide) conjugate (PC) or chondroitin alone at doses of 10 mg Pt/kg (platinum (IV)-polysaccharide) or 45 mg/kg (chondroitin). Tumor volumes and body weight were recorded daily for sixty days. Tumor volumes were measured as [length (l)×width (w)×thickness (h)]/2. Loss of body weight of 15% is considered a chemical-induced toxic effect. The results indicate that the platinum-polysaccharide conjugate (PC) is effective in vivo against breast tumor growth (FIG. 9). After treatment with platinum-polysaccharide conjugate, tumor tissues were dissected and embedded in formalin. The tumor tissue was fixed in paraffin, and stained with hematoxylin and eosin for histological examinations. Extensive necrosis was observed at 94 hrs post-administration of platinum-polysaccharide conjugate, but not polysaccharide alone (FIG. 10).

### Example 4

### Method of Tumor Cell Death or Inhibition

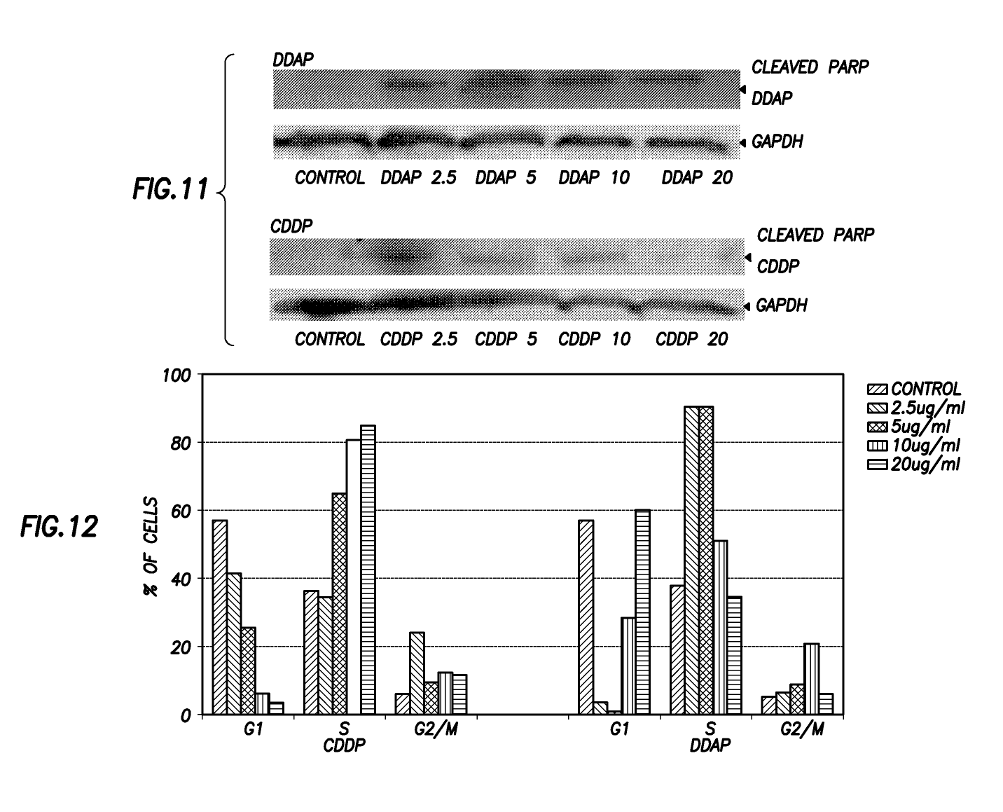

The effect of the platinum-polysaccharide conjugate of Example 1 on tumor cells was analyzed by treating 2008-c13 breast cancer cells with the conjugate, then analyzing the effects on cellular proteins through a Western blot (FIG. 11). Cleaved PARP was significantly increased in the cells treated with platinum-polysaccharide conjugate (PC), compared with cisplatin (CDDP), suggesting that platinum-polysaccharide conjugate inhibited 2008-c13 cell growth through enhancement of apoptosis in a caspase 3 dependent pathway.

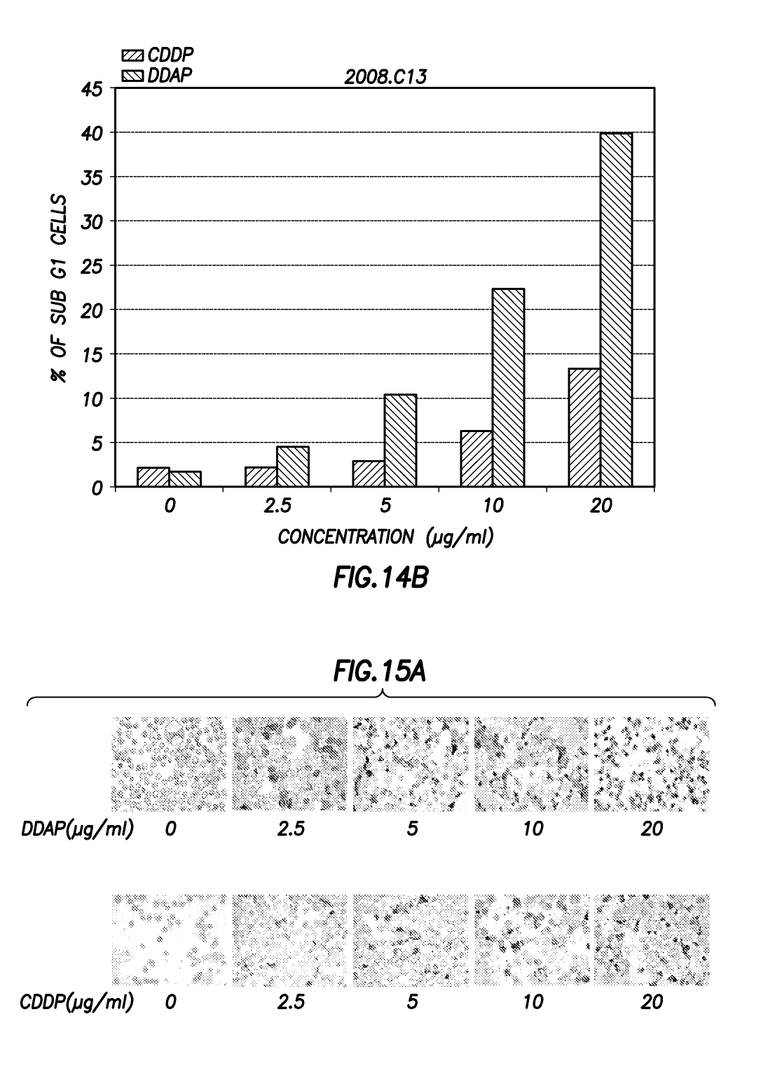

This effect was tested by flow cytometry in the 2008.C13 cell line after 48 hours and 72 hours of drug exposure. Flow cytometric analysis showed that there was a dose-dependent increase in the number of cells in the sub-G1 fraction after PC and CDDP treatments, which represents hypodiploid cells and indicates the induction of apoptosis. However, the use of PC, compared with CDDP, resulted in a more pronounced increase in the sub-G1 fraction at the same doses (FIG. 14).

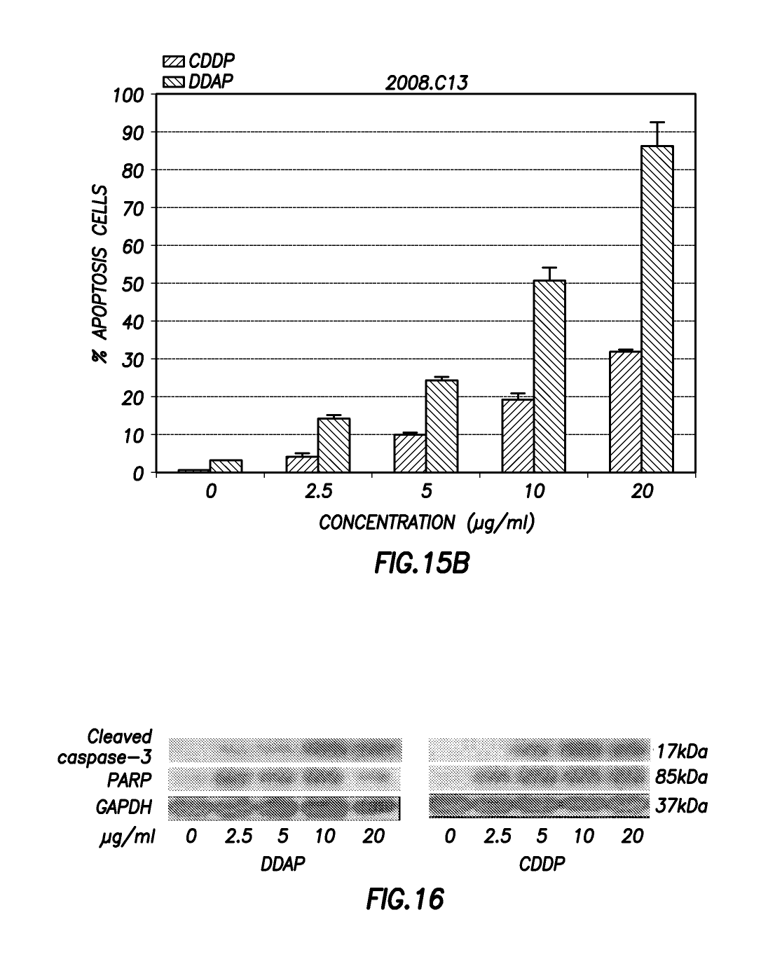

DNA fragmentation typical of apoptosis was further determined by the TUNEL assay in three independent experiments. A clear dose-dependent increase in the number of apoptotic cells was detected after exposure to both drugs. However, when compared at each dose, the PC-treated cells exhibited much higher levels of apoptosis (P<0.05) (FIG. 15).

To determine whether apoptosis is induced through a caspase-3-dependent pathway followed by the cleavage of PARP, levels of cleaved caspase-3 and PARP, which form after caspase-3 activation, were determined by Western blot analysis. PARP is a 113-kDa nuclear protein that has been shown to be specifically cleaved to an 85-kDa fragment during caspase-3-dependent apoptosis. After cells were exposed to CDDP or PC for 48 hours, cleaved PARP was present at each dose. In the CDDP-treated group, cleaved PARP expression increased from 2.5 μg/mL to 20 μg/mL and cleaved caspase-3 was expressed in a pattern similar to that of PARP. In the PC-treated group, the expression of cleaved caspase-3 was comparable to that in the CDDP-treated group, except for the lower expression seen at 5 μg/mL of PC. Although cleaved PARP expression induced by high-dose (20 μg/mL) PC appeared to be lower than that induced by low-dose PC, no such difference was detected in its upstream cleaved caspase-3 expression (FIG. 16).

In a further test on 2008-c13 breast cancer cells in vitro, flow cytometric analysis showed that cells significantly arrested in S-phase after exposure to platinum-polysaccharide conjugate (PC) at 48 hours (FIG. 12). The highest levels of S-phase blockage happened at lower dosages of 2.5 and 5 ug/ml (90.3% and 90.1%). When compared with cisplatin (CDDP), the effect of platinum-polysaccharide conjugate (PC) on arresting cells in S-phase is significantly different.

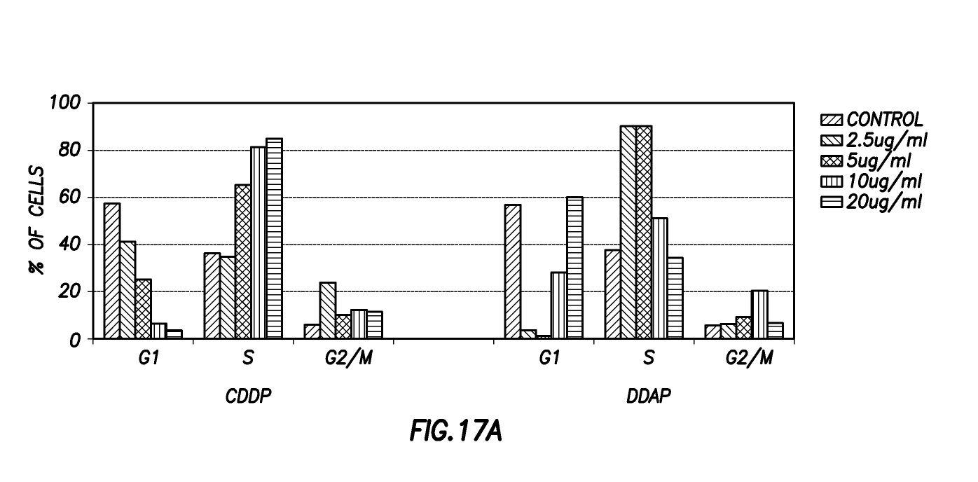

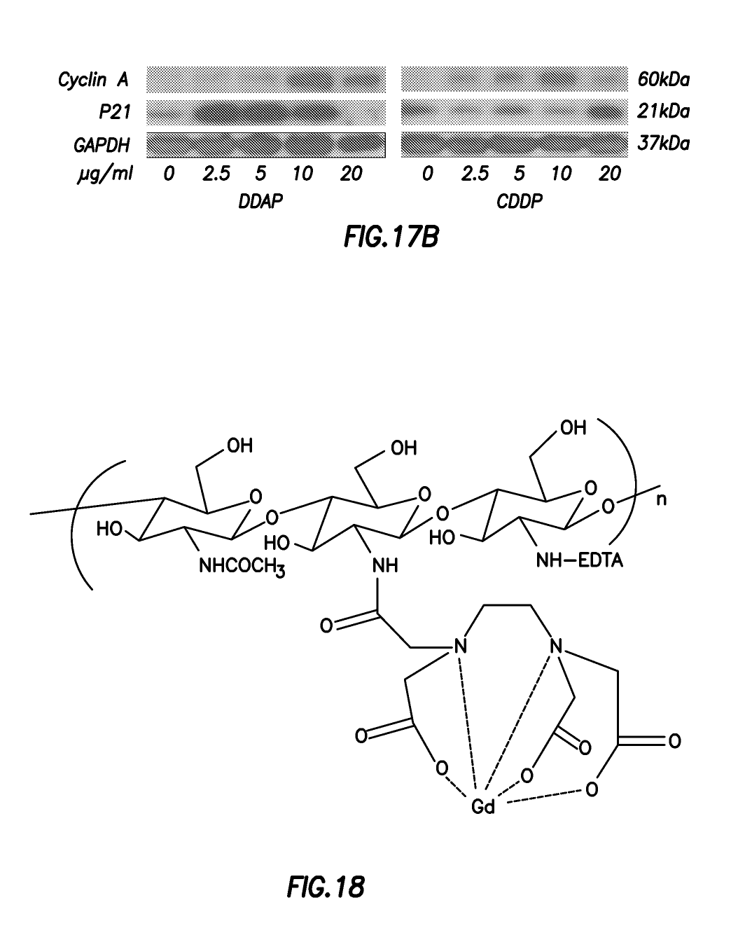

Specifically, DNA content was analyzed by flow cytometry 48 hours after 2008.C13 cells were treated with PC or CDDP. Exposure to CDDP induced cell arrest in the S-phase and increased the sub-G1 fraction at the 5 μg/mL dose, but not at the lowest dose, 2.5 μg/mL. The numbers of cells arrested in the S phase and sub-G1 fraction increased continuously as the CDDP dose increased, with the maximal S-phase arrest (84.8%) occurring at 20 μg/mL. After cells were exposed to PC for 48 hours, the highest levels of S-phase block occurred at the lower doses (2.5 μg/mL [90.3%] and 5 μg/mL [90.1%]). At higher doses (10 and 20 μg/mL), the level of S-phase arrest steadily decreased as the sub-G1 fraction increased. This can be explained by the fact that under the strong stress of high-dose PC, cells underwent apoptosis promptly and directly before they were arrested in the S-phase (FIG. 17).

To elucidate the mechanism underlying S-phase arrest caused by CDDP and PC in 2008.C13 cells, the expression of p21 and cyclin A, which are important for cell-cycle regulation in the S phase, was examined in 2008.C13 cells after 48 hours of drug exposure. Neither p21 nor cyclin A expression was related to the extent of S-phase arrest after CDDP treatment. After PC treatment, however, p21 and cyclin A expression were directly related to the extent of S-phase arrest: p21 was up-regulated with maximal S-phase arrest after low-dose PC treatment, but not after high doses; cyclin A was up-regulated after high-dose PC treatment and was maintained at a low level after low-dose PC treatment (FIG. 17B)

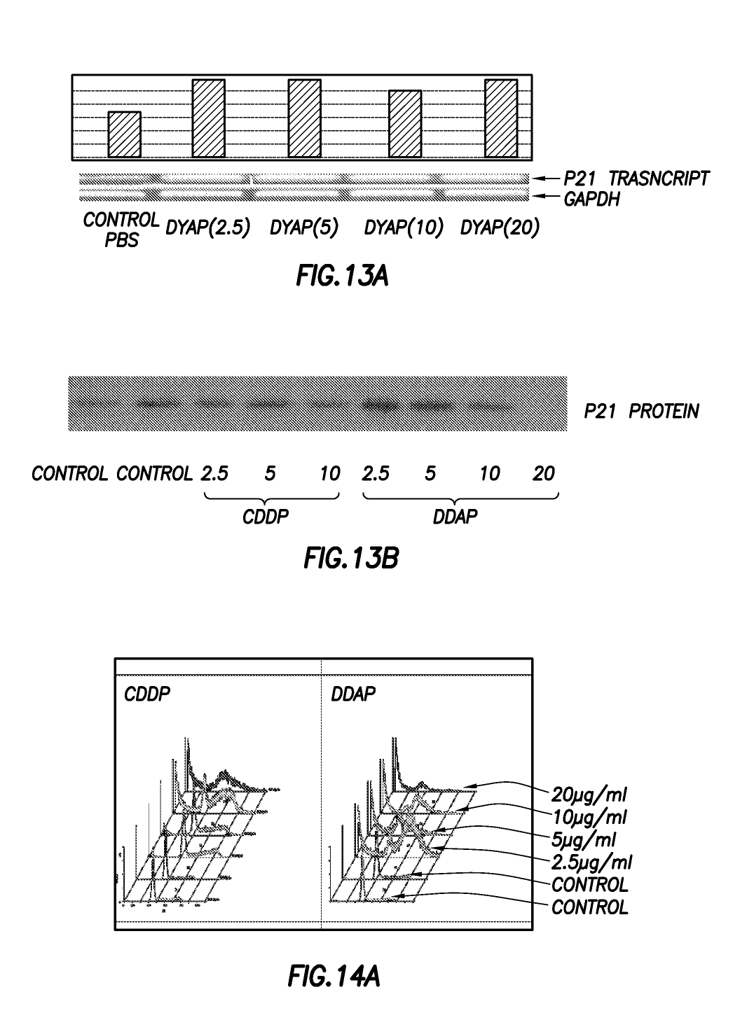

2008-c13 breast cancer cells treated with platinum-polysaccharide conjugate (PC) showed increased p21 expression at both transcriptional (FIG. 13A) and protein expression levels (FIG. 13B) as compared to cells treated with cisplatin (CDDP).

### Example 5

### Synthesis of Gd-EDTA-Chitosan (i.e. the Polysaccharide Conjugate of the Present Invention Materials

Chitosan (85% deacetylated and average molecular weight 3500 Da) was obtained from Dalwoo-ChitoSan, Korea (http://members.tripod.com/˜Dalwoo/index.htm). Ethylene diamine tetraacetic acid (EDTA) and dialysis membrane (M.W. cut off 3,500) were purchased from Fisher Scientific Company (Houston, Tex.). N-(3-dimethylaminopropyl)-N′-ethylcarbodiimide.HCL (EDC), Gd-DTPA and GdCl₃.6H₂O was purchased from Aldrich Chemical Company (Milwaukee, Wis.).

Synthesis of Gd-EDTA-chitosan was done in two steps as described below.

### Step 1. Synthesis of EDTA-Chitosan.

2.59 gm of chitosan was dissolved in 259 mL of water. Then 8.1 gm (27 7 mmol) ethylenediamine tetraacetic acid (EDTA) was added to it. The pH of the solution was adjusted to 6 with 5 M NaOH. After adjusting pH, 5.31 gm (27.7 mmol) of EDC was added to the above solution and pH was adjusted to 6 again. The reaction mixture was stirred overnight at room temperature. The volume of the bulk reaction mixture was reduced by lyophilization and then it was dialyzed against water with a dialysis membrane (MWCO 3500) for 36 hours. The dialyzed solution was frozen, and lyophilized to get about 8.5 gm of EDTA-chitosan. Without any further purification, the crude product was used for Gadolinium chelation.

### Step 2. Chelation of Gd With EDTA-Chitosan.

5.4 gm of EDTA-chitosan was dissolved in 45 mL of water. A solution of gadolinium chloride with a concentration of 100 mg/mL was prepared and added dropwise to the EDTA-chitosan solution, and pH was maintained between 6 to 6.5 with 0.5N NaOH. GdCl₃ addition was continued until the EDTA-chitosan solution began to turn turbid. The reaction mixture was allowed to stir for about 4 hours at room temperature and then centrifuged to isolate insoluble solid from the water. The pH of the clear solution of Gd-EDTA-chitosan was made to 8 and then dialyzed against water with a dialysis membrane (MWCO 3500) for 36 hours and it was freeze dried to yield 2.7 gm of Gd-EDTA-chitosan. No free Gd was detected using 4-(2-Pyridylazo)-resorcinol as an indicator. From elemental analysis the Gd content was found 20.6%(w/w). The structure of Gd-EDTA-chitosan is shown in FIG. 18.

### Example 6

### In Vitro Determination of Relaxivity

Solutions of Gd-EDTA-chitosan and Gd-DTPA were prepared in water at Gd concentrations of 0.01 mM, 0.02 mM, 0.04 mM and 0.08 mM. Spin lattice (T₁) relaxivity was measured at 7.0 T. R₁ in mM⁻¹s⁻¹ was obtained from linear last-squares determination of the slopes of 1/T₁ vs [Gd] plot. R₁ was calculated as 3.61 and 9.59 mMs⁻¹ for Gd-DTPA and Gd-EDTA-chitosan, respectively.

### Example 7

### In Vivo Animal Studies Using MRI

MRI studies were performed in two models. For tumor-bearing rat model, rats were inoculated with 13762 breast tumor cells at hind legs (sc, 10,000 cells/rat). When the tumor reached 1.5-2 cm, the MRI studies were conducted. Breast tumor-bearing rats were anesthetized using 2-4% and 1-2% isoflurane in oxygen, respectively. Animals were placed head first and prone on an imaging sled, with anesthetic gas delivered through a nosecone and a respiratory bellows taped around the abdomen to allow monitoring of the physiological state of the animals. Two-dimensional axial images were obtained using a 7.0 T/30 cm MR scanner (Bruker Biospin Corp., Billerica, Mass.) with 400 mT/m, 12-cm inner diameter actively shielded gradient coil system (2,760 T/m/s slew rate) and a 72-cm inner diameter volume radiofrequency coil. Sagittal T2-weighted RARE scans (TE/TR 60 ms/3000 ms, field of view 8 cm x 6 cm, matrix size 256 x 128, 2 mm slices, 2 averages) were used to visualize the position of the tumor. Axial T₁-weighted spin-echo scans (TE/TR 9 ms/1000 ms, FOV 6 cm×6 cm, 256×256 matrix, 2 mm slices) and T₂-weighted RARE scans (TE/TR 60 ms/3000 ms, FOV 6 cm×6 cm, 256×256 matrix, 2 mm slices, 4 averages) of the tumor were acquired with matching slice prescriptions. A fast spoiled gradient echo (FSPGR) sequence (TE/TR 1.7 ms/60 ms, FOV 6 cm×6 cm, 128×96 matrix, 2 mm slices, 45 degree excitation angle, 80 repetitions) was used to continuously acquire T₁-weighted images every 5.8 seconds from approximately 1 minute before to 6.5 minutes after injection of Gd-DTPA and Gd-EDTA-chitosan (0.14 mmol/kg). Following completion of the dynamic FSPGR acquisition, axial T₁-weighted spin-echo scans (as described above) were repeated for one hour. The pixel-by-pixel signal intensity (SI) values for tumor and for normal muscle (in the contralateral leg) were measured using regions of interest (ROIs) defined using both the precontrast T₁-weighted and the T₂-weighted image data. For each animal, three contiguous images, representing the largest tumor cross-sectional slices, were analyzed. For each section, the tumor area was computed from the total number of pixels contained in each ROI and the pixel dimension. Also in each imaging section, the signal intensity enhancement within the ROI was expressed as the ratio of SI values between tumor and normal muscle tissue.

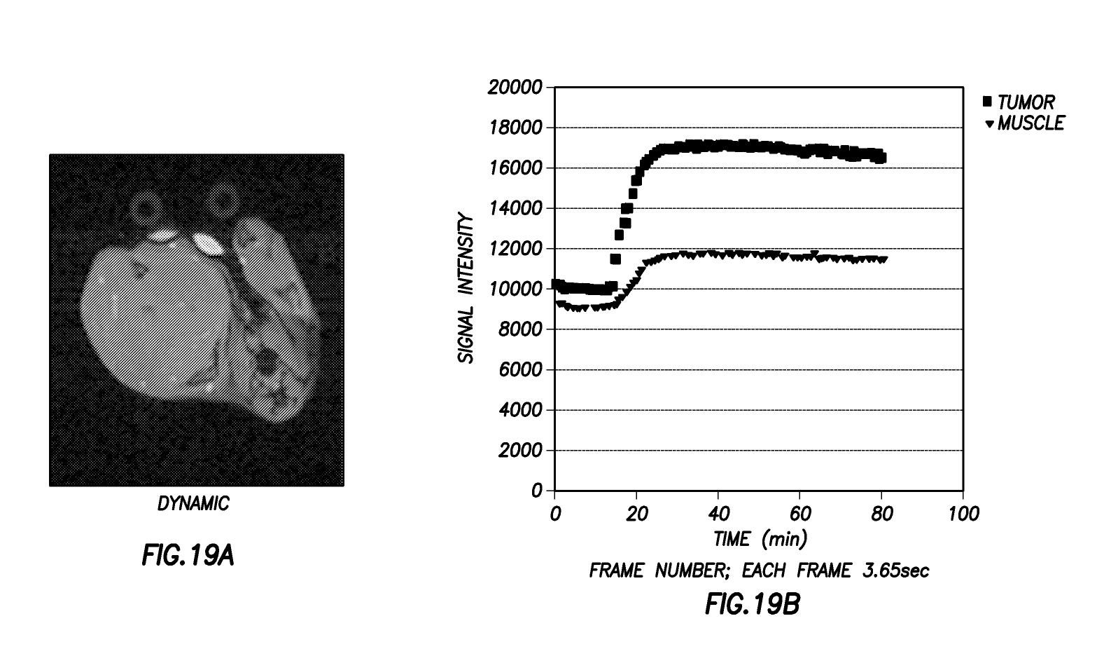

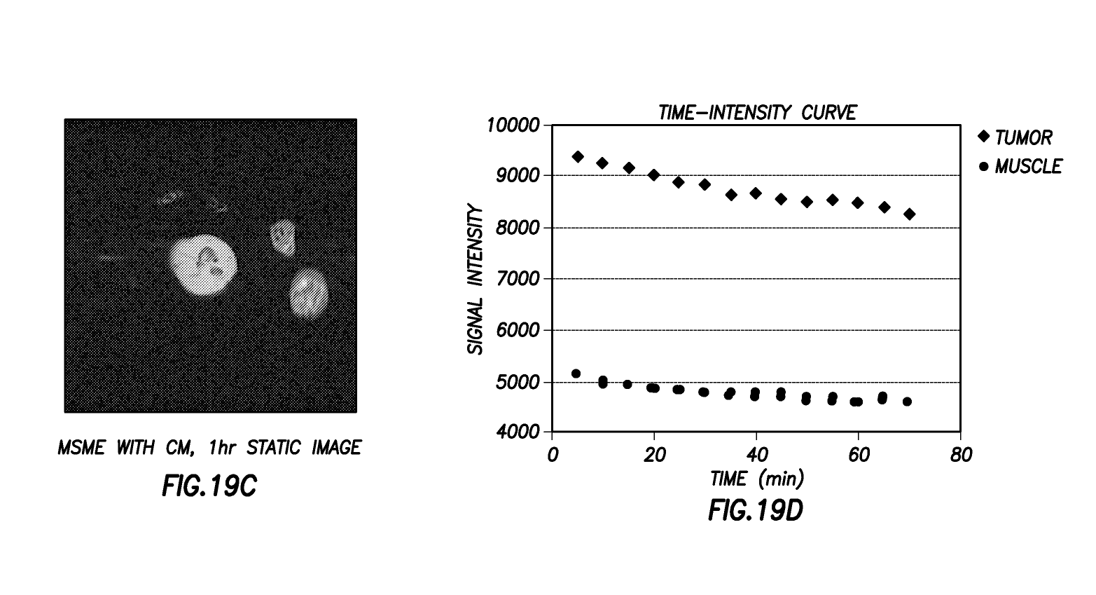

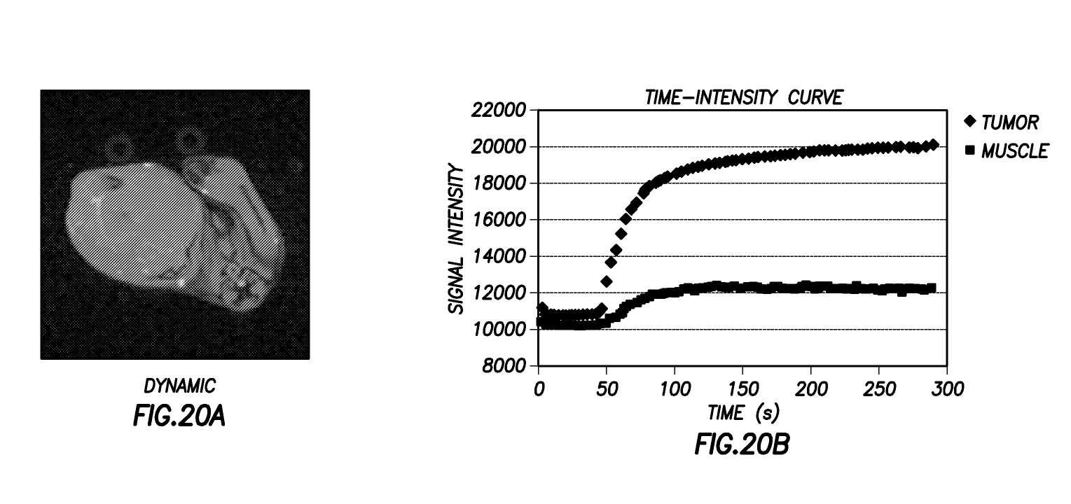

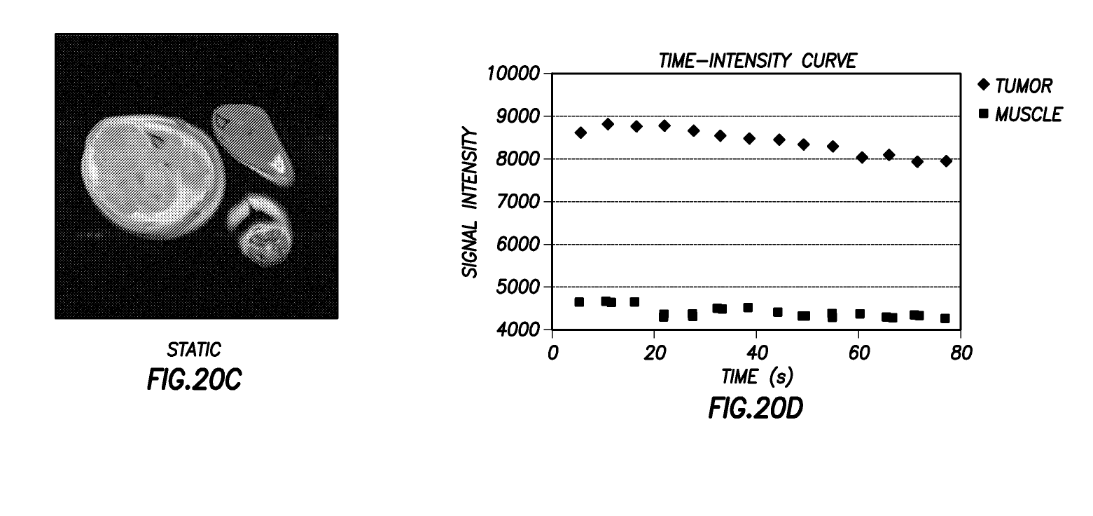

FIG. 19 shows the results of In Vivo Animal Studies using MRI after injection of Gd-EDTA-chitosan. FIG. 20 shows the results of In Vivo Animal Studies using MRI after injection of Gd-DTPA. FIGS. 19 and 20 exhibited that Gd-DTPA and Gd-EDTA-chitosan were similar in the signal intensity of tumors; however, the relaxivity in vitro of Gd-EDTA-chitosan was higher than that of Gd-DTPA.

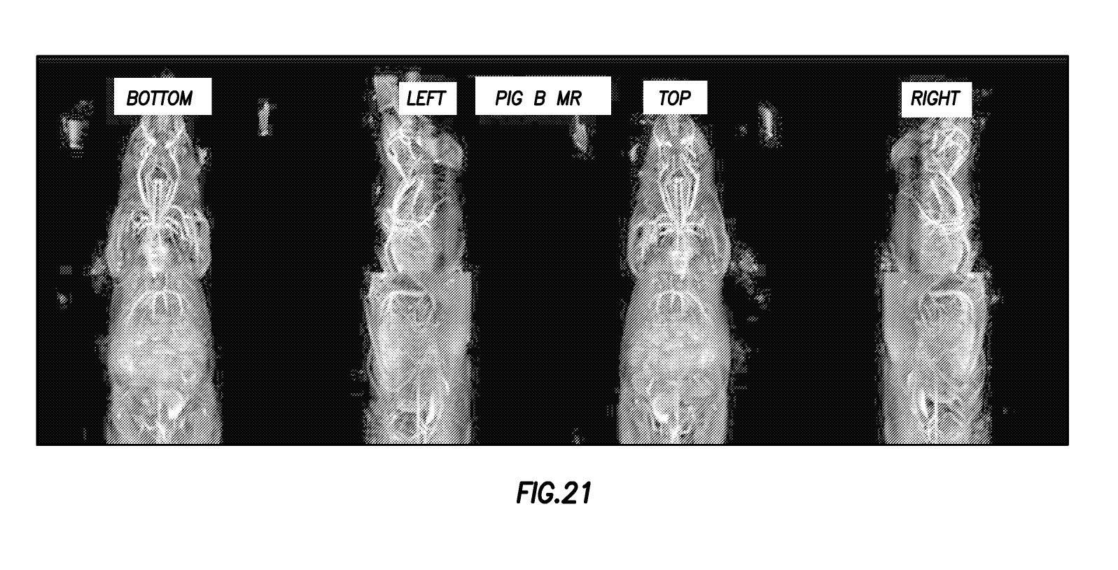

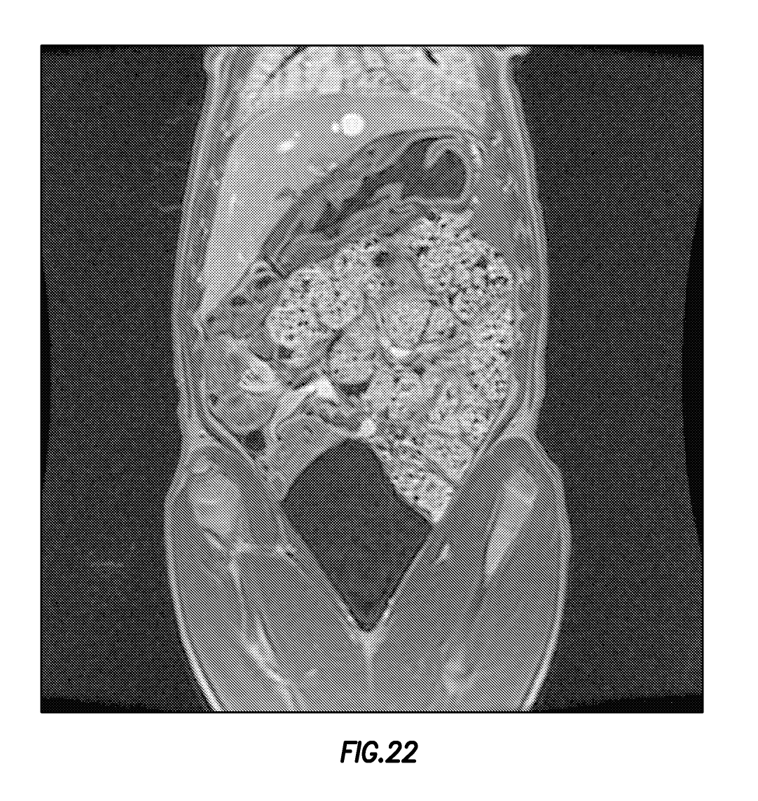

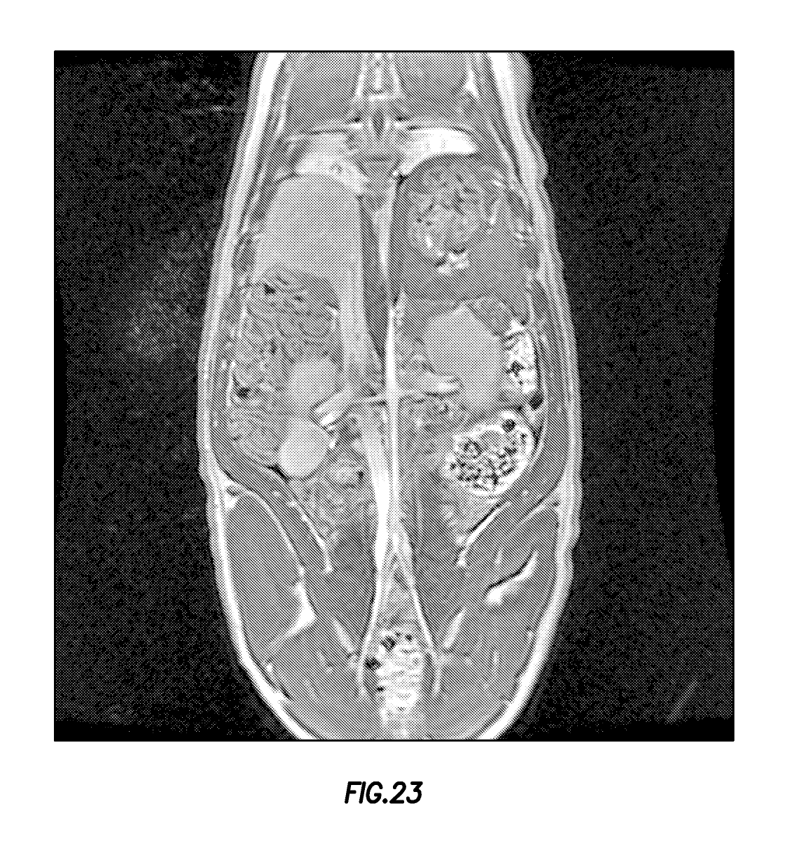

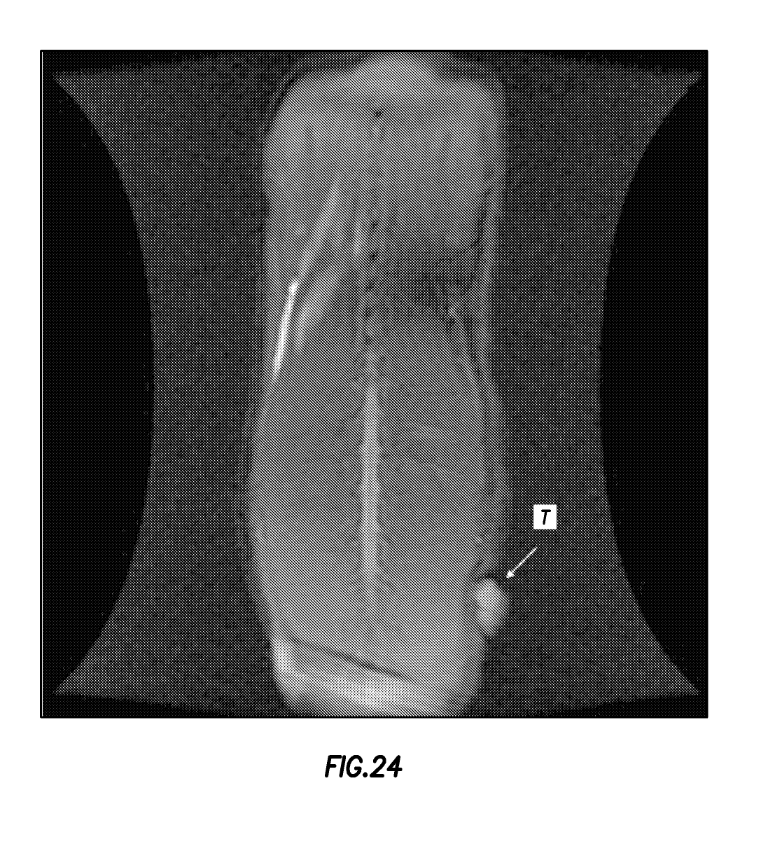

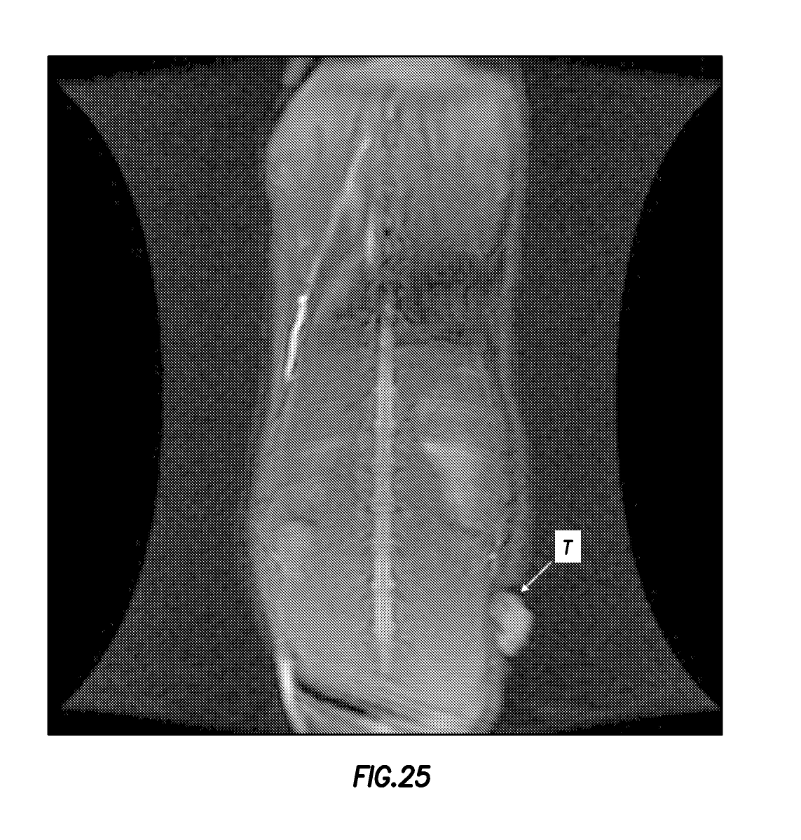

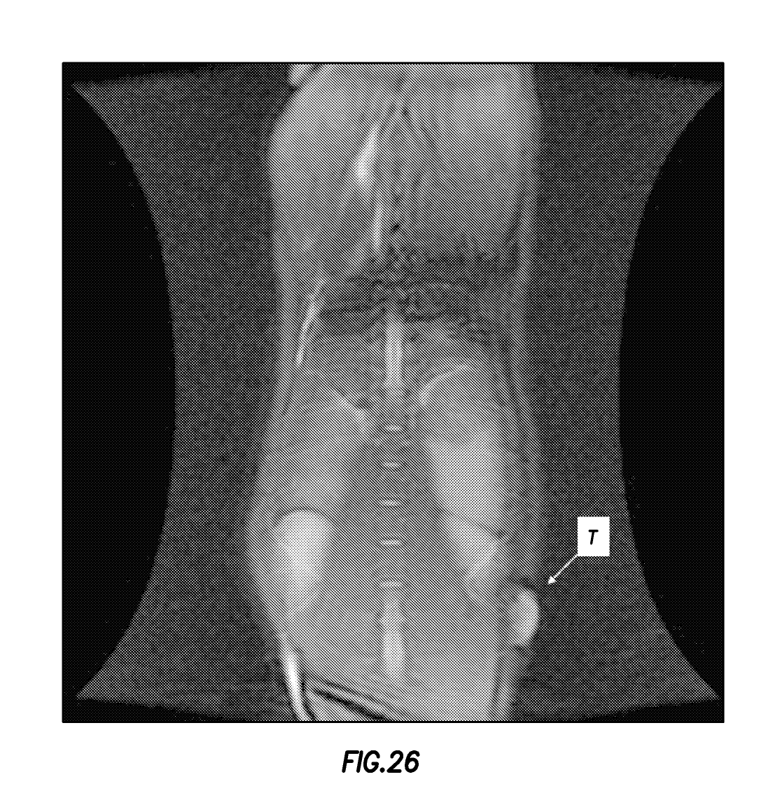

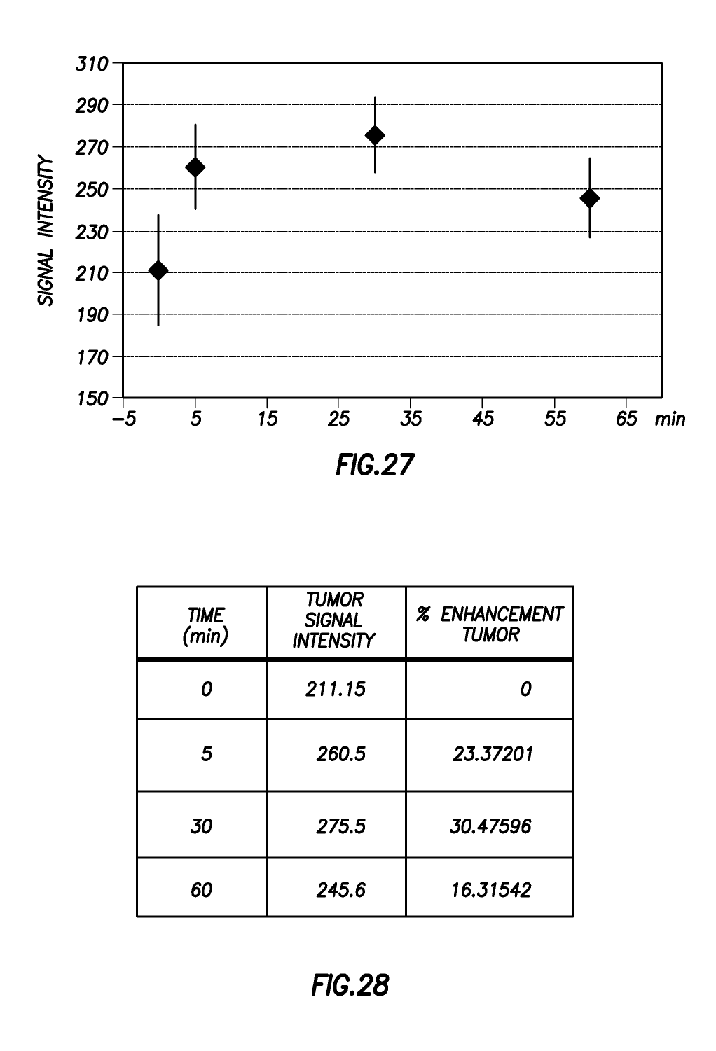

For a normal pig (20 kg), the images were acquired at 15 min after the dose of Gd-EDTA-chitosan (0.13 mmol/kg, iv). For melanoma-bearing pig, the pig (23 kg) was positioned supine. MRI scanning was performed at baseline and repeated at 5, 30, and 60 min after the dose of Gd-EDTA-chitosan (0.13 mmol/kg, iv). All functional MRI data were acquired by using GE 1.5-T EchoSpeed scanners (256×128 matrix; ten 5-mm slices acquired every 11.04 seconds for 5.53 minutes; 22- to 36-cm axial field of view). Acquired images were sent from each site to a central image-analysis vendor (VirtualScopics, LLC, Rochester, N.Y.) for quality assessment and final analysis. Signal-intensity time curves were generated by averaging all voxels in each region of interest at each time point Curves normalized by subtracting the average precontrast signal intensity for each region of interest. MRI of a normal pig administered with Gd-EDTA-chitosan at 15 min post-injection revealed vascular targeting, as shown in FIG. 21-23. MRI of a melanoma-bearing pig administered Gd-EDTA-chitosan at 5, 30, and 60 min post-injection revealed that the tumor could be imaged. The results of MRI of a melanoma-bearing pig administered Gd-EDTA-chitosan at 5, 30, and 60 min post-injection were shown in FIGS. 24, 25, and, 26, respectively. Time-activity curve of tumor uptake (signal intensity) versus time of Gd-EDTA-chitosan in a melanoma-bearing pig was shown in FIG. 27. According to FIG. 27, the maximal signal intensity was at 30 min post-injection of Gd-EDTA-chitosan. The signal intensity in FIG. 27 was further converted into the percentage (%) of tumor enhancement and the converted results were shown in FIG. 28. FIG. 28 indicated that the percentage (%) of tumor enhancement was ranged 16.3-30.5%, compared to precontrast signal intensity. The optimal enhancement was at 30 min post-injection of Gd-EDTA-chitosan.

### Example 8

### Planar Imaging Studies

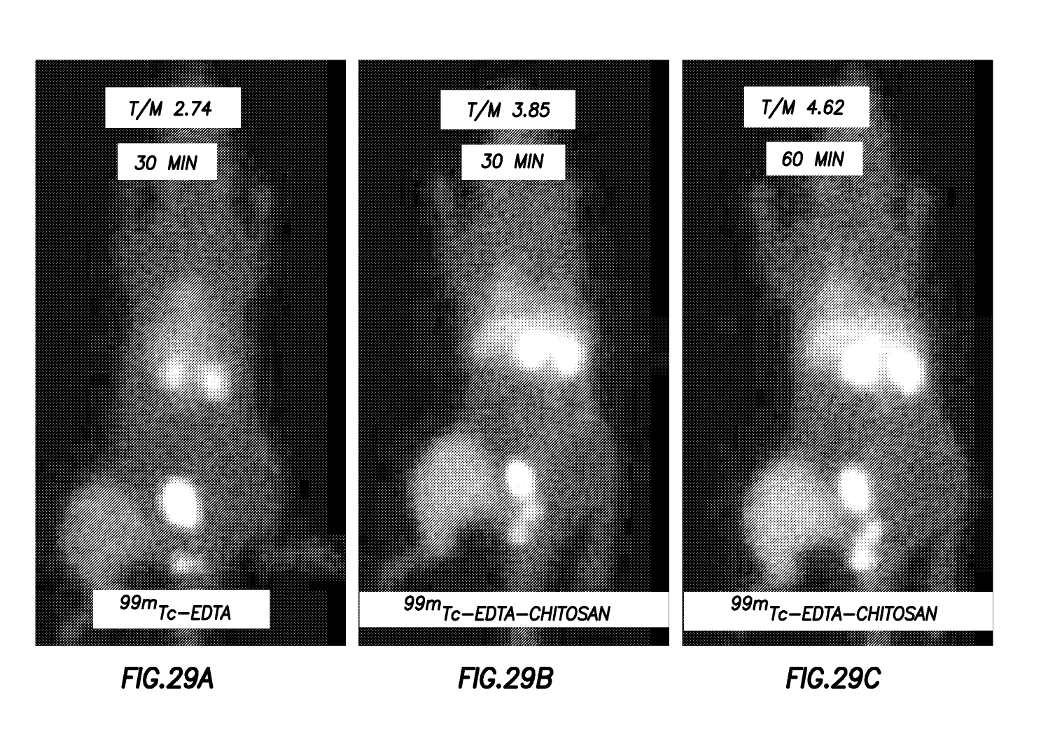

To demonstrate Gd-EDTA-chitosan can be applied to a hybrid camera such as nuclear scan or MRI, EDTA-chitosan was labeled with^{99m}Tc in the presence of tin (II) chloride. Fischer 344 rats were inoculated subcutaneously with mammary tumor cells (10⁵ cells/rat) at hind legs. On day 14 post-inoculation, mammary tumor-bearing rats were imaged with ^{99m}Tc-EDTA-chitosan (1 mg/rat) and ⁹⁹mTc-EDTA (3 mg/rat) (300 μCi/rat, iv) at 0.5 and 1 hr using M-camera (Siemens) acquired at 500,000 counts. Computer outlined regions of interest (ROI) (counts per pixel) of tumor lesion site and symmetric normal muscle site were used to determine tumor-to-background count density ratios. The planar scintigraphy of breast tumor-bearing rats after treatment with ^{99m}Tc-EDTA or ^{99m}Tc-EDTA-chitosan for 0.5 or 1 hr was shown in FIG. 29. Planar scintigraphy of breast tumor-bearing rats at 0.5 hr showed that the tumor/muscle uptake ratio in ^{99m}Tc-EDTA-chitosan was higher than ^{99m}Tc-EDTA (3.85 vs 2.74, respectively).

### Example 9

### Application of Gd-EDTA-Chitosan for Computed Tomography (CT)

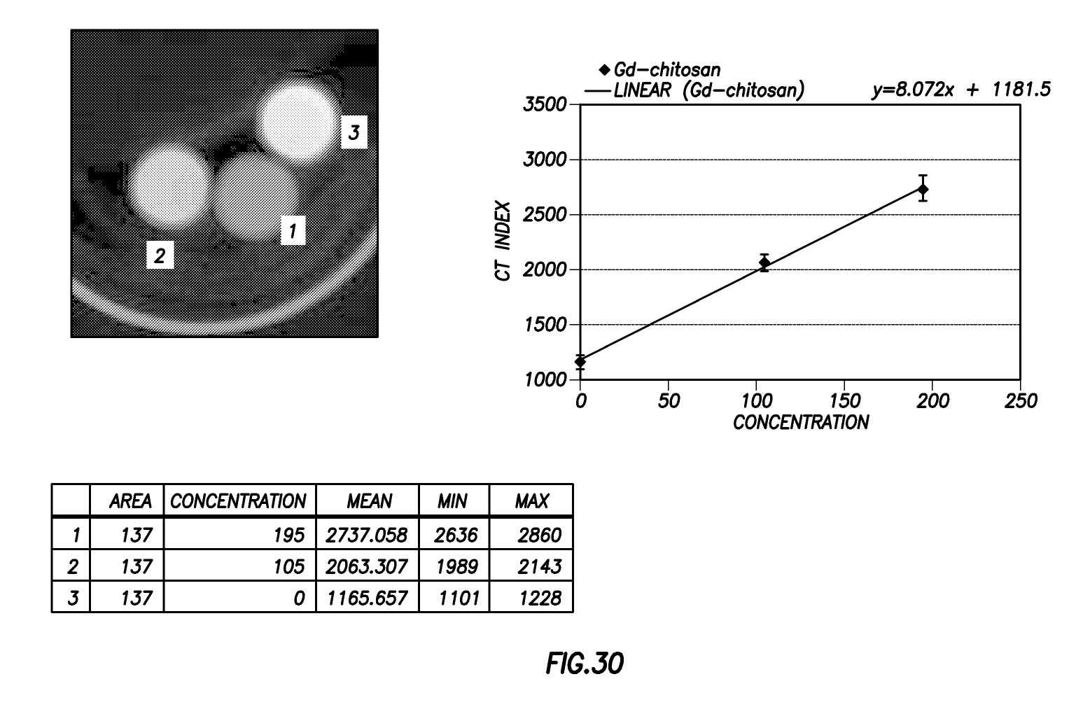

To demonstrate Gd-EDTA-chitosan can be applied to CT due to the nature of metal effect such as Gd, two concentrations (100 and 200 mM) of Gd-EDTA-chitosan were prepared. A phantom assay of these concentrations was preformed by CT. The results were shown in FIG. 30. The results indicated that the higher concentration of Gd-EDTA-chitosan (i.e. number 3) revealed sufficient intensity, compared to pre-contrast (i.e. number 1). The results also proved that Gd-EDTA-chitosan is able to be applied to CT.

### Example 10

### Application of Gd-EDTA-Chitosan for Thermotherapy and Neutron Capture Therapy

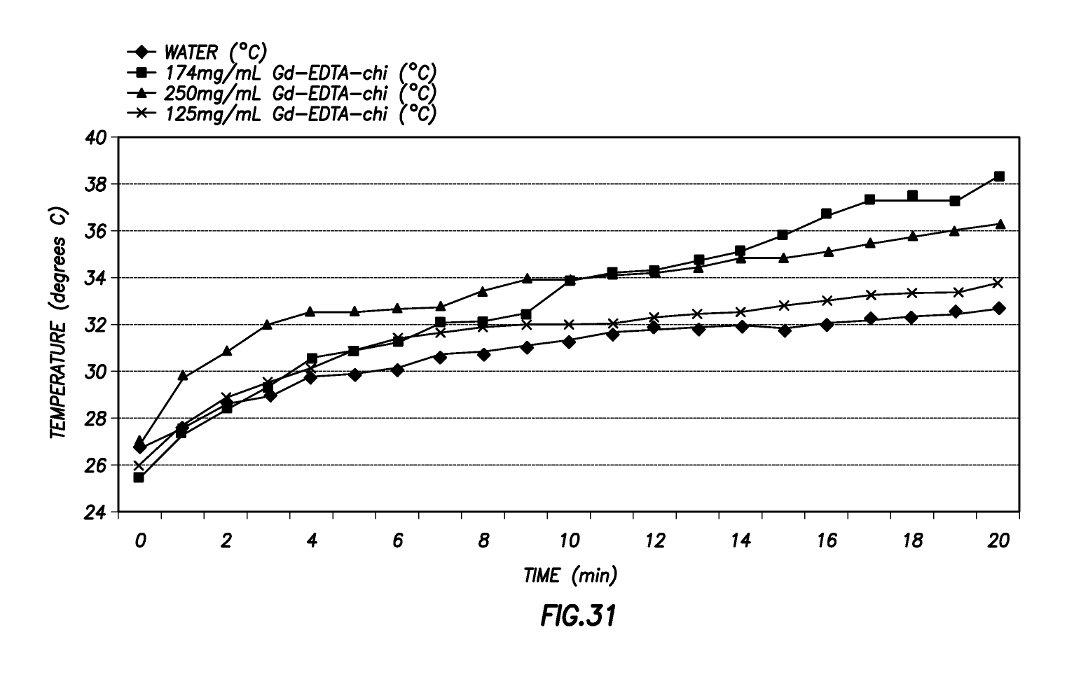

The Gd-EDTA-chitosan elemental analysis determined there was 20.6% w/w gadolinium loading. At various concentrations of Gd-EDTA-chitosan (125-250 mg/mL, Gd-eq 25-50 mg/mL), compared to Gd-DTPA (174 mg/mL, Gd-eq 50 mg/mL), changes of temperatures were achieved by Gd-EDTA-chitosan using ultrasound bath at 40 KHz (FIG. 31). The technology platform can quantify tumor uptake of Gd-EDTA-chitosan by MRI which allows Gd-157(n,gamma)/Gd-158 reaction for neutron capture therapy (NCT).

### Example 11

### Synthesis of Au/Ag Composite-Thiol Compound-Chitosan Conjugate

Au/Ag composite-thiol compound-chitosan conjugate was synthesized by reaction of thiol compound-chitosan conjugate with Au/Ag composite, wherein the thiol compound-chitosan conjugate was obtained by reaction of chitosan with 2-iminothiolane and the synthesis of the Au/Ag composite was disclosed in Taiwanese patent application No. 100111493. The synthesis of Au/Ag composite-thiol compound-chitosan conjugate was described in detail as below.

10 mg of chitosan was dissolved in 1 mL of deionized water with well stirring until there was complete dissolution of chitosan to obtain aqueous solution of chitosan. Then, 2 mg of 2-iminothiolane was added to the aqueous solution of chitosan and reacted at room temperature for 3 hours to obtain the aqueous solution of chitosan modified with thiol group (i.e. thiol compound-chitosan conjugate), which was used without further purification.

Because there is a good binding ability between thiol group and Au, Au/Ag composite-thiol compound-chitosan conjugate can be easily synthesized by mixing the thiol compound-chitosan conjugate with Au/Ag composite. To 10 mL of mixture of 10 mM HAuCl₄ and 10 mM AgNO₃ solution, 4 μL of 0.1M ascorbic acid (reductant) was added. After reaction for 1 min, 1 mL of the aqueous solution of thiol compound-chitosan conjugate described as above was added to obtain a growth solution. Then, the growth solution was placed steadily for 1 min to obtain Au/Ag composite-thiol compound-chitosan conjugate.

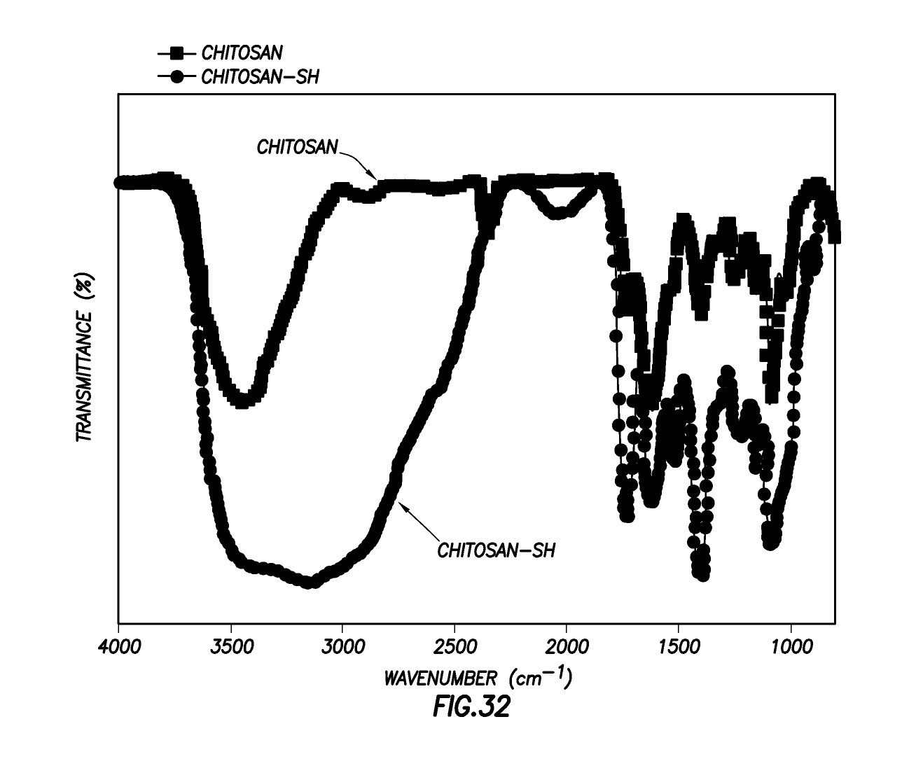

FIG. 32 shows the images of Fourier transform infrared spectrum of chitosan and thiol compound-chitosan conjugate. The results indicated that the images of FTIR of chitosan and thiol compound-chitosan conjugate were similar in transmittance and both had peaks of 3425 cm⁻¹ (N—H), 1630 cm⁻¹ ((N)C═O), 2920 cm⁻¹ (C—H), and 1098 cm⁻¹ (C—O). Besides, the image of FTIR of the thiol compound-chitosan conjugate had a peak of 3119 cm⁻¹ (S—H). The peak at 3119 cm⁻¹ was resulted from the reaction of 2-iminothiolane with the functional group (amino group) of the end of chitosan, which implies that thiol compound-chitosan conjugate was successfully synthesized.

In summary, the polysaccharide conjugate of the present invention exhibits prolonged blood circulation and preferential tumor accumulation due to a different blood vessel permeability between normal tissue and solid tumor. Furthermore, the polysaccharide conjugate of the present invention is useful for the assessment of tumor angiogenesis by MRI. Below are the highlights of Gd-EDTA-chitosan.

Although only exemplary embodiments of the invention are specifically described above, it will be appreciated that modifications and variations of these examples are possible without departing from the spirit and intended scope of the invention.

1. A polysaccharide conjugate comprising:
a polysaccharide;
at least one linker covalently bound to the polysaccharide; and
at least one metal conjugated by said linker.

2. The polysaccharide conjugate of claim 1, wherein the polysaccharide has a molecular weight of between 3,000 daltons and 30,000 daltons.

3. The polysaccharide conjugate of claim 1, wherein the polysaccharide is selected from the group consisting of: collagen, chondroitin, hyauraniate, chitosan, and chitin.

4. The polysaccharide conjugate of claim 1, wherein the polysaccharide comprises chitosan.

5. The polysaccharide conjugate of claim 1, wherein the polysaccharide has an amino group and the linker is covalently bound to the polysaccharide via said amino group.

6. The polysaccharide conjugate of claim 1, wherein the linker has a carboxyl group or thiol group, and said metal is conjugated by the carboxyl group or thiol group of the linker.

7. The polysaccharide conjugate of claim 1, wherein the linker is a chelating agent or a modifier.

8. The polysaccharide conjugate of claim 7, wherein the chelating agent is ethylene diamine tetraacetic acid.

9. The polysaccharide conjugate of claim 7, wherein the linker is the open-ring form of the modifier.

10. The polysaccharide conjugate of claim 9, wherein the modifier is iminothiolane.

11. The polysaccharide conjugate of claim 1, wherein the metal is selected from the group consisting of: Tc-99m, Cu-60, Cu-61, Cu-67, In-111, Tl-201, Ga-67, Ga-68, As-72, Re-188, Ho-166, Y-90, Lu-177, Sm-153, Sr-89, Gd-157, Gd-158, Bi-212, Bi-213, Fe, Au, Ag, and composite of Au and Ag.

12. The polysaccharide conjugate of claim 1, wherein the polysaccharide conjugate comprises the polysaccharide ranged from 50% to 80% by weight.

13. The polysaccharide conjugate of claim 1, wherein the polysaccharide conjugate comprises the linker ranged from 10% to 40% by weight.

14. The polysaccharide conjugate of claim 1, wherein the polysaccharide conjugate comprises the metal ranged from 10% to 30% by weight.

15. A method of synthesizing a polysaccharide conjugate comprising:
covalently bonding a linker to a polysaccharide to obtain an intermediate; and
conjugating said intermediate to a metal to form a polysaccharide conjugate.

16. The method of claim 15, further comprising drying the polysaccharide conjugate to form a powder.
